SEQUENCE LISTING (16 sequences)

SEQ ID NO: 1 - protein, 305 aa, Streptomyces antibioticus
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 2 - protein, 307 aa, Streptomyces Sp. Strain Cl190 [sequence omitted]

SEQ ID NO: 3 - protein, 305 aa, Actinobacteria bacterium OV320
MSGAADVERVYAAMEEAAGLLDVSCAREKIYPLLTVFQDTLTDGVVVFSMASGRRSTELDFSISVPVSQGDPYATVVREGLFRATGSPVDELLADTVKHLPVSMFAIDGEVTGGFKKTYAFFPTDDMPGVAQLTGIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSPTEEYYKLGAYYHITDIQRQLLKAFDALED

SEQ ID NO: 4 - protein, 305 aa, Streptomyces tendae
MSGAADVERVYAAMEEAAGLLDVSCAREKIYPLLTVFQDTLTDGVVVFSMASGRRSTELDFSISVPVSQGDPYATVVKEGLFRATGSPVDELLADTVKHLPVSMFAIDGEVTGGFKKTYAFFPTDDMPGVAQLTEIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSSTEEYYKLGAYYHITDIQRQLLKAFDALED

SEQ ID NO: 5 - protein, 305 aa, Streptomyces sp. Root1310
MSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRQLLKAFDALED

SEQ ID NO: 6 - protein, 305 aa, Streptomyces paucisporeus
MSGAAEVERVYSAMEEAAGLLDVACSPEKVRPILTAFQDVLSDGVIVYSMASGRHATELDFSISVPADHGDPYTAALAHGLIPETDHPVGNLLADTQKALPVSMFAVDGEVTGGFKKTYAFFPTDDMPGLAQLIDIPSMPPSVAENAELFARYGLDKVQMTSLDYKRKQVNLYFSNLQPEFLAPEPVLSMVREMGLELPGEKGLKFARRSFAIYPTLGWESGKIERLCFAVISTDPGLVPAPDEADRALFSTYANNAPYAYAGEKRTLVYGLTLSPTEEYYKLGSYYQITDIQRTLLKAFDALTD

SEQ ID NO: 7 - protein, 305 aa, Streptomyces sp. URHA0041
MSGAAEVERVYSAMEESAGLLDVACSREKIQPILTAFQDVLADGVIVFSMANGRHATELDFSISVPAGHGDPYAAALEHGLIPATGHPVGDLLADTQKALPVSMFAVDGEVTSGFKKTYAFFPTDDMPGLAQLIDIPSMPPSVAENAELFGRYGLDKVQMISLDYKKNQVNLYFSNLNPEFLQPEPVQAMVREMGLQLPADKGLAFAKRSFAVYPTLSWDSAKIERLCFAVISTDPTLAPAQEQADLDLFSTYANNAPYAYAGEKRTLVYGLTLSPSEEYYKLGSYYQISDIQRKLLKAFDALTD

SEQ ID NO: 8 - protein, 305 aa, Streptomyces sp. CNQ-509
MSGAADVERVYSAMEEAARLLDITVSREKVRPALEAYHEVLADAVVVFSMASGRYATELDFSISVPAEAGDPYRVALAKGLTPRTDHPVGRLLADTQEHCPVSMFAFDGEITGGFKKTYAFFPTNDLQSASKLAEIPSMPDSVKENADLFARYGLDKVQMTSIDYKKKAVNLYFSEMSPDILGPDTVRSMLRDMGLKETGETGLTFARRSFSVYPTLNWETGRIERLCFAVISRDPTLAPAERAEDLAKFSKYANNAPYAYAGEARTLVYGLTLTPREEYYKLGSYYQISDIQRKLLKAFDSLND

SEQ ID NO: 9 - protein, 304 aa, Unknown
  Bacterial
MSGAKDVERVYSAMEEAAGLLNVPVARDKIWPVLTAYQDALADAVIVFSMAGGRRSTELDFSISVPTDHGDPFTTALERGLTEKENHPVDNLLAELRDGFPLGMYAIDGMVTTGFKKAYASFPTNEPQPLTALLDLPSMPESARANAELFARYGLDKVQMVSVDYPKRQVNLYFSELKADHLTPEQVKATASEMGLVEPTDMALDFATGSFAVYPTLGYDSDVVDRITYAVISVDPTLAPTTSEPEKTQITTYANSAPYAYAGENRTLVYGFTLTSKEEYYKLGSYYQITDLQRTLVKAFEALD

SEQ ID NO: 10 - protein, 304 aa, Streptomyces sp. CNH099
MSGAKDVERVYSAMEEAAGLLNVPVARDKIWPVLTAYQDALADAVIVFSMAGGRRSTELDFSISVPTDHGDPFTTALERGLTEKENHPVDNLLAELRDGFPLGMYAIDGMVTTGFKKAYASFPTNEPQPLTALLDLPSMPESARANAELFARYGLDKVQMVSVDYPKRQVNLYFSDLNADHLTPEEVKSTASEMGLVEPTDMALDFATGSFAVYPTLGYDSDVVDRITYAVISVDPTLAPTTSEPEKTQITTYANSAPYAYAGENRTLVYGFTLTSKEEYYKLGSYYQITDLQRTLVKAFEALD

SEQ ID NO: 11 - protein, 304 aa, Unknown
  Bacterial
MSGANDVERVYSAMEEAAGLLNVPVARDKIWPVLTAYQDALADAVVVFSMAGGRRATELDFSISVPTDLGDPFTTALRRGLTEKTNHPVDNLLAELTDGFEIGMYAIDGMVTTGFKKTYASFPTNEPQPLTALLDVPSMPESARANAELFARYGLDKVQMVSVDYPKRQVNLYFSELDTDYLQPEHVKSLARETGLVEPTEMGLDFASGSFAVYPTLGYDNDIVDRITYAVISVDPTLAPTKSEPEVSQLSRYATSAPYAYAGENRTLVYGVTLTSKEEYYKLGSYYQITDLQRTLVKAFEALD

SEQ ID NO: 12 - protein, 304 aa, Unknown
  Bacterial
MSGANDVERVYSAMEEAAGLLGVPVAREKVRPVLTAYQDALADAVVVFSMAGGRRATELDFSISVPTDHGDPFTTALQRGLTEKTGHPVDNLLAELREGFPLGMYAIDGMVSTGFKKTYASFPTNEPQPLDDLLDVPSMPASARANAKLFANYGLDKVQMVSVDYPKRQVNLYFSELNTDYLQPAQVKALAAEMGLIEPSELGLEFAKGSFAVYPTLSYDTDASDRLCLAVISSDPTLAPTTSEPEVTQFSTYANNAPYAYAGENRTLVYGLTLTPKEEYYKLGSYYQITDYQRKLVKAFEALD

SEQ ID NO: 13 - protein, 306 aa, Streptomyces sp. 303MFCol5.2
MSKATEVDRVYAAVEKAAALAGTTCAGDKVRPVLTGHQDLLDEAVIVFSMTASGSHSGGLDLSMTVPAEHVDPYSFALSEGLIEPTDHPVGSVISDFQERFPIGMYGIDVDVAGGFKKAYAAFPSNDLRELKQLFDLPSMPSAAAENAELFARYGLDRVTGVSVDYKRHELNLYCDRATTEPLDPDYVQSMLRDMGLKEASEQGLEFAKKTFAIYPTLNWDSSEIVRICFAVITTDPATTPTRSEPELGQMWEYANTAPYAYVGEQRALVYGLALSPEKEYYKLGAYYQISDYQRKLVKAFDALPE

SEQ ID NO: 14 - protein, 378 aa, Streptomyces niveus
MCVPGSRARRPGSRGWLERTAKPAPTRGTVGAKVRSQTWERRAPGATTVTCPVQGRSTGPIQADIQDRHVGDSMSGAADVERVYSAMERAAGLLDLTCAREKILPILTAYKEALADSVIVFSMSGGDHSAELDFSFTIPSGDVDPYAFGPSTGIPTETDHPIASLLSDTGERCPVAMYGVDGEVSGGFKKTYAAFPINDLLDLSKLVAVPSMPPAVAENAELFARYGLDKVQGISIDYQRKQVNLYCGDIPAESLEPETVRSMLREMGLREPSEEGLEFVRKSFAVYPTLSWDSSRIERICFAVISTDPTLAPTRVESDVALFSKYANNAPYAYAGERRTLIYGLAVSPTKEYIKLGSYYQISDHQRKLVKAFDALED

SEQ ID NO: 15 - protein, 311 aa, Streptomyces sp. CNH287
MYGGTEVEEVYSALEKSAGLVGVPCNRDKVWPALSTYQDALGEAVIVFSVATDERHAGELDYTITVPTGGADPYALALAKGLTPETDHPVGTLLAGVQERCPVAGYAVDCGVVGGFKKIYSFFPQDDLQGLAKLAEIPSMPRALAENAALFARHGLDHKVTMLGIDYQRESVNLYFGKLPEECLQPDSIRAILRDIGLPEPTEPMLEFARKSFAIYVTLSWDAAKVERICFAVPPGRDLITLDPSALPARIAPEIEHFARNSPYAYPGDRMLVYGVTWSPEEEYYKLGSYYQLPVQTRKLLVAFDSVKDQE

SEQ ID NO: 16 - protein, 305 aa, Artificial Sequence
  Prenyltransferase variant
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAVYHITDIQRRLLKAFDALED

(19) United States
(12) Patent Application Publication
(10) Pub. No.: US 2022/0347192 A1
(43) Pub. Date: Nov. 3, 2022

(54) PRENYLTRANSFERASE VARIANTS AND METHODS FOR PRODUCTION OF PRENYLATED AROMATIC COMPOUNDS

(71) Applicant: Genomatica, Inc., San Diego, CA (US)

(72) Inventors: Michael A. Noble, San Diego, CA (US); Kevin G. Hoff, San Diego, CA (US); Russell S. Komor, San Diego, CA (US)

(21) Appl. No.: 17/640,196

(22) PCT Filed: Sep. 4, 2020
(86) PCT No.: PCT/US2020/049430

§ 371 Date: Mar. 3, 2022

Related U.S. Application Data
(60) Provisional application No. 62/897,174, filed on Sep. 6, 2019.

(51) Int. Cl. A61K 31/60; C12N 1/20; C12N 15/52; C12P 7/42; C12P 17/06; A61K 31/05; A61K 31/352; A61K 35/742; A61K 35/74; A61K 36/062; A61K 36/02

(52) U.S. Cl. CPC A61K 31/60; C12Y 205/01102; C12N 1/205; C12N 15/52; C12P 7/42; C12P 17/06; A61K 31/05; A61K 31/352; A61K 35/742; A61K 35/74; A61K 36/062; A61K 36/02; C12N 2800/101; C12R 2001/19

(57) ABSTRACT

Described herein are non-natural variants of prenyltronsfcrases having at least one amino acid substitution as compared to its corresponding natural or unmodified prenyltransferascs. The variants are capable of an increased rate of formation of prenylated aromatic compounds, such as cannabinoids, as compared to a wild type control The prcnyltransferase variants can be expressed in an engineered microbe having a pathway to such cannabinoids, and optionally can include one or more other pathway transgencs to promote formation of substrate(s) for the prcnyltransferases. Therapeutically useful cannabinoids can be purified from engineered cells and cell cultures.

### PRIORITY CLAIM

This application claims the benefit of U.S. Provisional Patent Application Ser. No. 62/897,174 filed Sep. 6, 2019, entitled PRENYL TRANSFERASE VARIANTS AND METHODS FOR PRODUCTION OF PRENYLATED AROMATIC COMPOUNDS, the disclosure of which is incorporated herein by reference. Also, the entire contents of the ASCII text file entitled “GNO0116WO_Sequence_Listing.txt” created on Sep. 3, 2020, having a size of 43 kilobytes is incorporated herein by reference.

### BACKGROUND

Cannabinoids constitute a varied class of chemicals that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses.

Cannabis sativa produces a variety of phytocannabinoids, the most notable of which is a precursor of tetrahydrocannabinol (THC), the primary psychoactive compound in cannabis. However, C. sativa also produces precursors of other cannabinoids such as cannabidiol (CBD), cannabigerol (CBG), and cannabichromene (CBC). CBD, CBG, and CBC, which, unlike THC, are not psychoactive. In C. sativa, precursors of CBD, CBG, CBC, and THC, are carboxylic acid-containing molecules referred to as Δ⁹-tetrahydrocannabinoic acid (Δ⁹-THCA), CBDA, cannabigerolic acid (CBGA), and cannabichromenic acid (CBCA), respectively. Δ⁹-THCA, CBDA, CBGA, and CBCA are bioactive after decarboxylation, such as caused by heating, to their bioactive forms, e.g. CBGA to CBG.

Despite the well-known actions of THC, the non-psychoactive CBD, CBG, and CBC cannabinoids also have important therapeutic uses. For example, these cannabinoids can be used for the treatment of conditions and diseases that are altered or improved by action on the CB₁ and/or CB₂ cannabinoid receptors, and/or α₂-adrenergic receptor. CBG has been proposed for the treatment of glaucoma as it has been shown to relieve intraocular pressure. CBG can also be used to treat inflammatory bowel disease. Further, CBG can also inhibit the uptake of GABA in the brain, which can decrease anxiety and muscle tension. Cellular synthesis of CBG, via CBGA, derives from olivetolic acid and geranyldiphosphate pathways. Formation of olivetolic acid stems from fatty acid biosynthesis in which hexanoic acid is produced and which in turn is converted to hexanoyl-CoA through hexanoyl CoA synthetase. Polyketide synthase catalyzes three sequential condensation reactions of malonyl-CoA onto hexanoyl-CoA to form 3,5,7-trioxododecanoyl-CoA which is converted to olivetolic acid (2,4-dihydroxy-6-pentylbenzoate) by the enzyme olivetolic acid cyclase (Gagne et al., PNAS, 109: 12811-12816). Formation of geranyldiphosphate stems from the mevalonate pathway (MVA) or methylerythritol-4-phosphate pathway (MEP; also known as the deoxyxylulose-5-phosphate), which produce isopentyl pyrophosphate (IPP) and dimethylallyl pyrophosphate (DMAPP), which are converted to geranyl pyrophosphate (GPP) using geranyl pyrophosphate synthase.

Geranyl-pyrophosphate-olivetolic acid geranyltransferase (EC 2.5.1.102, GOT) catalyzes the following reaction:

geranyl diphosphate+2,4-dihydroxy-6-pentylbenzoate→diphosphate+cannabigerolic acid

The enzyme carrying out the above reaction in C. sativa is a transmembrane prenyltransferase belonging to the UbiA superfamily of membrane proteins. See for example WO2011017798A1 describing CsPT1. However, the above reaction has also been reported to be carried out by a different family of enzymes. In particular, aromatic prenyltransferases that are soluble, non-transmembrane, and have a 10-stranded antiparallel β-barrel consisting of 5 repeated αββα motifs, can catalyze the transfer of isoprenoid chains to aromatic rings. For example, Yang, Y., et al. (Biochemistry 51:2606-180 (2012)) reports that NphB, a Streptomyces-derived, soluble enzyme, catalyzes the attachment of a 10-carbon geranyl group to aromatic substrates; originally identified in the biosynthetic pathway of the antioxidant naphterpin. Yang notes the reaction mechanism of the prenylation step has been characterized as a S(N)1 type dissociative mechanism with a weakly stable carbocation intermediate. NphB catalyzes the prenyl transfer between GPP and 1,6 dihydroxynaphthalene (1,6-DHN) and yields three products with the geranyl moiety attaching to different carbon atoms of 1,6-DHN. The major product 5 geranyl DHN and minor product 2-geranyl DHN were characterized with a product ratio of 10:1.

A subsequent publication Kumano et. al (Bioorg. Med. Chem, 16:8117-8126 (2008)), reports rates and regioselectivity measurements for NphB-catalyzed geranylation of olivetol, with mixed regioselectivity at 2- and 4-OL ring positions, and rates of 0.0026 mol 2-geranyl-OL/min/mol NphB and 0.0016 mol 4-geranyl-OL/min/mol NphB, which are extremely slow.

### SUMMARY

Aspects of the disclosure are directed towards forming prenylated aromatic compounds, including cannabinoids, engineered enzymes (e.g., prenyltransferase variants of the soluble aromatic prenyltransferase type) with improved activity that facilitate cannabinoid formation, non-natural cells including the engineered enzymes and prenylated aromatic compound formation, including cannabinoid pathways, fermentation methods using the same, and improved prenylated aromatic compound preparations, including cannabinoid product preparations. In particular, disclosure associated with the current invention is directed towards non-natural prenyltransferases that include at least one amino acid variation that differs from an amino acid residue of a wild type soluble type prenyltransferase.

Non-natural prenyltransferases of the disclosure promote formation of prenylated alkylbenzenediols or prenylated dihydroxyalkylbenzenoic acids from a substrate comprising a hydrophobic portion (such as dimethylallyl pyrophosphate, geranyl pyrophosphate, farnesyl pyrophosphate, and geranylgeranyl pyrophosphate), and alkylbenzenediols or dihydroxyalkylbenzenoic acids, respectively. Non-natural prenyltransferases of the disclosure also demonstrate regioselectivity towards desired products, for example, the variants are capable of regioselectivity (e.g., about 90% or greater) to 2-prenylated 5-alkylbenzene-1,3-diol or 3-prenylated 2,4-dihydroxy 6-alkylbenzenoic acid from geranyl pyrophosphate and a 5-alkylbenzene-1,3-diol, or a 2,4-dihydroxy 6-alkylbenzenoic acid, respectively.

In experimental studies associated with the invention, non-natural prenyltransferase variants were identified that demonstrated improved activity for catalyzing the reaction between olivetolic acid (OLA) and geranyl diphosphate (GPP) to form the product 3-geranyl-olivetolate (cannabigerolic acid; CBGA, 3-GOLA). Non-natural prenyltransferase variants were also identified that demonstrated improved activity for catalyzing the reaction between orsellinic acid (OSA) and geranyl diphosphate (GPP) to form the product cannabigerorcinic acid (CBGOA).

The non-natural prenyltransferase variants of the disclosure have at least one amino acid substitution as compared to its corresponding natural prenyltransferases of the soluble, αββα (ABBA) structural type, or a prenyltransferases having one or more variations that are different than one or more variations that provide improved activity and/or regioselectivity to 3-GOLA. In some preferred aspects, non-natural prenyltransferase variants have two variant amino acid substitutions, or preferable three or four variant amino acid substitutions. For example, a prenyltransferase with a different mutation which may have been previously engineered can be used as a template, prior to incorporating any modification described herein. Such prenyltransferases that are starting sequences for incorporating a modification described herein to generate the novel engineered enzyme may be alternatively referred to herein as wild-type, template, starting sequence, natural, naturally-occurring, unmodified, corresponding natural prenyltransferases, corresponding natural prenyltransferases without the amino acid substitution, corresponding prenyltransferases or corresponding prenyltransferases without the amino acid substitution(s). Experimental studies described demonstrate that a number of amino acid positions along the length of the prenyltransferase sequence can be substituted to provide non-natural prenyltransferases having increased activity and desired regioselectivity. Experimental studies associated with the disclosure show single substitutions and combinations of substitutions in a prenyltransferase template can provide increased activity and desired regioselectivity, and therefore provide single and combination variants of a starting or template or corresponding prenyltransferases, e.g., in particular enzymes of the class EC 2.5.1.102, having increased substrate conversion and/or regio selectivity.

Non-natural prenyltransferase variants can be based on known prenyltransferase proteins, homologs thereof, variants thereof, or even enzymatically active fragments thereof. The non-natural prenyltransferase variant of the disclosure can be based on the soluble ABBA type prenyltransferase from Streptomyces antibioticus AQJ23_40425 (NCBI Accession number KUN17719.1; 305 amino acids long; SEQ ID NO: 1), or a homolog thereof. In some embodiments, the non-natural prenyltransferase variant of the disclosure is based on homologs of SEQ ID NO: 1, such as the prenyltransferase homologs represented by any of SEQ ID NOs 2-15. The variant can also be based on a prenyltransferase having a certain amount of sequence identity to any of SEQ ID NOs: 1-15, such as 50% or greater identity, 60% or greater identity, etc. The locations of one or more variant amino acids can be described with reference to one or more amino acid positions in a SEQ ID NO, such as SEQ ID NO: 1, or a corresponding position in one of SEQ ID NOs: 2-15, based on alignment with SEQ ID NO: 1. Exemplary non-natural prenyltransferase variants of the disclosure have about 55% or greater identity, about 60% or greater identity, about 65% or greater identity, about 70% or greater identity, about 75% or greater identity, about 80% or greater identity, about 85% or greater identity, about 87.5% or greater identity, about 90% or greater identity, about 92.5% or greater identity, about 95% or greater identity, about 98% or greater identity, about 99% or greater identity, or 100% identity to any of SEQ ID NOs: 1-15, and prenyltransferase activity. Also included are second generation variants, for example, a previously identified prenyltransferase variant such as SEQ ID NO: 16 which has variant amino acids at positions 159, 212, and 286 relative to SEQ ID NO:1, and further comprising one or more other amino acid variations of the disclosure.

In one embodiment, the disclosure provides a non-natural prenyltransferase having 50% or greater identity to any one of SEQ ID NOs:1-16, comprising at least one amino acid variation as compared to a wild type prenyltransferase, wherein the at least one amino acid variation is selected from a variation at position: 45; 121, wherein the position 121 amino acid variation is 121V; 124, wherein the position 124 amino acid variation is selected from 124K and 124L; 160; 173; 212, wherein the position 212 amino acid variation is 212N; 232, wherein the position 232 amino acid variation is selected from 232K, 232N, 232R, and 232S; 267, wherein the position 267 amino acid variation is selected from 267A and 267P; 269, wherein the position 269 amino acid variation is 269F; 286, wherein the position 286 amino acid variation is 286W; 290; 294; 296, wherein the position 296 amino acid variation is selected from 296K, 296M, and 296Q; 300, wherein the position 300 amino acid variation is 300Y; wherein the amino acid locations are relative to SEQ ID NO:1 or a corresponding amino acid location in any of SEQ ID NOs:2-16.

In another embodiment, the disclosure provides a non-natural prenyltransferase having 50% or greater identity to any one of SEQ ID NOs:1-15, comprising at least three amino acid variations as compared to a wild type prenyltransferase, wherein at least two of the amino acid variations are selected from variations at locations 159, 212, and 286, and at least one other amino acid variation is selected from a variation at position 45, 47, 49, 121, 124, 160, 173, 213, 230, 232, 267, 269, 290, 294, 296, and 300, the amino acid locations relative to SEQ ID NO:1 or a corresponding amino acid location in any of SEQ ID NOs:2-15

In some embodiments, in the non-natural prenyltransferase of the disclosure the one or more amino acid variation(s) at a position selected from the group consisting of 45, 121, 124, 160, 173, 212, 230, 232, 267, 269, 286, 290, 294, 296, and 300 is selected from the group consisting of: 45I; 45T; 45S, 121V; 124K; 124L; 160L; 160V; 160I; 173D; 173K; 173P; 173Q; 173E; 173F; 212N; 230S; 232K; 232N; 232R; 232S; 267A; 267P; 269F; 286W; 290A; 290I; 290M; 290S; 294K; 294H; 296K; 296M; 296Q; and 300Y.

In some embodiments, in the non-natural prenyltransferase of the disclosure the one or more amino acid variation(s) at a position selected from the group consisting of 45, 121, 124, 160, 173, 212, 230, 232, 267, 269, 286, 290, 294, 296, and 300 is selected from the group consisting of: 45I; 45T; 121V; 124K; 124L; 160L; 173D, 173K, 173P, 173Q; 212N; 230S; 232K; 232N; 232R; 232S; 267A; 267P; 269F; 286W; 294K; 296K; 296M; 296Q; and 300Y.

In some embodiments, the non-natural prenyltransferase can have 50% or greater identity to any one of SEQ ID NOs:1-15, and can include (a) at least two amino acid variations selected from 159S, 212H, and 286V, such as a prenyltransferase having 159S and 286V variations, or a prenyltransferase having 159S, 212H, and 286V variations.

In embodiments, in addition to the one or more variant amino acids described herein, the non-natural prenyltransferase can further comprise one or more other amino acid variation(s) not specified and selected from a variation at one or more position(s) selected from the group consisting of 45, 47, 49, 121, 124, 160, 173, 213, 230, 232, 267, 269, 290, 294, 296, and 300. For example, the non-natural prenyltransferase can further comprise one or more amino acid variation(s) selected from the group consisting of: 124S; 232T; 296I; 300P, and 300I.

In embodiments, in addition to the one or more variant amino acids described herein, the non-natural prenyltransferase can further comprise one or more other amino acid variation(s) at one or more position(s) selected from the group consisting of 121, 161, 175, 211, 214, 230, 268, 269, 284, 285, 286, 288, 293, 295. For example, the non-natural prenyltransferase can further comprise one or more amino acid variation(s) selected from the group consisting of: 47S, 47N, 47G; 121L; 161R, 161H, 161S; 175H, 175K, 175R; 211H; 211N; 214H; 230S; 268Y; 269N; 284S; 285Y; 286F, 286L, 286M, 286P, 286T, 286V, 286I, 286A; 288V, 288I; 296I; 293H, 293M, 293F, 293W, 293C, 293C, 293A, 293S, 293V, 293D, 293Y, 293E, 293I, and 293T.

Exemplary non-natural prenyltransferases can have at least three amino acid variations at the following positions relative to SEQ ID NO:1 or a corresponding amino acid location in any of SEQ ID NOs:2-15, with the variations selected from: (i) 45I, (ii) 159S, (iii) and 286V; (i) 45T, (ii) 159S, and (iii) 286V; (i) 121V, (ii) 159S, and (iii) 286V; (i) 124K, (ii) 159S, and (iii) 286V; (i) 124L, (ii) 159S, and (iii) 286V; (i) 159S, (ii) 160L and (iii) 286V; (i) 159S, (ii) 160L and (iii) 286V; (i) 159S, (ii) 160S and (iii) 286V; (i) 159S, (ii) 173D and (iii) 286V; (i) 159S, (ii) 173K and (iii) 286V; (i) 159S, (ii) 173P and (iii) 286V; (i) 159S, (ii) 173Q and (iii) 286V; (i) 159S, (ii) 173Y and (iii) 286V; (i) 159S, (ii) 212H and (iii) 286V; (i) 159S, (ii) 230S and (iii) 286V; (i) 159S, (ii) 267P and (iii) 286V; (i) 159S, (ii) 286V; and (iii) 293H; (i) 159S, (ii) 286V; and (iii) 294K; (i) 159S, (ii) 286V; and (iii) 296K; (i) 159S, (ii) 286V; and (iii) 296L; (i) 159S, (ii) 286V; and (iii) 296M; (i) 159S, (ii) 286V; and (iii) 296Q; (i) 159S, (ii) 286V; and (iii) 296M; (i) 159S, (ii) 286V; and (iii) 300F; and (i) 159S, (ii) 286V; and (iii) 300Y.

Other exemplary non-natural prenyltransferases can have at least four amino acid variations at the following positions relative to SEQ ID NO:1 or a corresponding amino acid location in any of SEQ ID NOs:2-15, with the variations selected from: (i) 45I, (ii) 159S, (iii) 212H, and (iv) 286V; (i) V45T, (ii) 159S, (iii) 212H, and (iv) 286V; (i) 121V, (ii) 159S, (iii) 212H, and (iv) 286V; (i) 124K, (ii) 159S, (iii) 212H, and (iv) 286V; (i) 124L, (ii) 159S, (iii) 212H, and (iv) 286V; (i) 159S, (ii) 160L, (iii) 212H, and (iv) 286V; (i) 159S, (ii) 160L, (iii) 212H, and (iv) 286V; (i) 159S, (ii) 160S, (iii) 212H, and (iv) 286V; (i) 159S, (ii) 173D, (iii) 212H, and (iv) 286V; (i) 159S, (ii) 173K, (iii) 212H, and (iv) 286V; (i) 159S, (ii) 173P, (iii) 212H, and (iv) 286V; (i) 159S, (ii) 173Q, (iii) 212H, and (iv) 286V; (i) 159S, (ii) 173Y, (iii) 212H, and (iv) 286V; (i) 159S, (ii) 212H, (iii) 213V, and (iv) 286V; (i) 159S, (ii) 212H, (iii) 230S, and (iv) 286V; (i) 159S, (ii) 212H, (iii) 267P, and (iv) 286V; (i) 159S, (ii) 212H, (iii) 286V, and (iv) 293H; (i) 159S, (ii) 212H, (iii) 286V, and (iv) 294K; (i) 159S, (ii) 212H, (iii) 286V, and (iv) 296K; (i) 159S, (ii) 212H, (iii) 286V, and (iv) 296L; (i) 159S, (ii) 212H, (iii) 286V, and (iv) 296M; (i) 159S, (ii) 212H, (iii) 286V, and (iv) 296Q; (i) 159S, (ii) 212H, (iii) 286V, and (iv) 296M; (i) 159S, (ii) 212H, (iii) 286V, and (iv) 300F; and (i) 159S, (ii) 212H, (iii) 286V, and (iv) 300Y.

The non-natural prenyltransferases of enzymatically-promoted formation of 2-prenylated, 5-alkylbenzene-1,3-diols or 3-prenylated, 2,4-dihydroxy 6-alkylbenzenoic acids from geranyl pyrophosphate from 5-alkylbenzene-1,3-diols, or 2,4-dihydroxy 6-alkylbenzenoic acids at greater rates than corresponding wild type enzymes. For example, non-natural prenyltransferases of the disclosure are enzymatically capable of (a) greater than about 1.5-fold rate of formation of 3-geranyl-olivetolate (3-GOLA), analogs, derivatives, or combinations thereof from geranyl pyrophosphate (GPP) and olivetolic acid (OLA), analogs, derivatives, or combinations thereof, as compared to the corresponding wild type prenyltransferase; (b) greater than about 1.5-fold rate of formation of 3-geranyl-orsellinate (3-GOSA from geranyl pyrophosphate (GPP) and orsellinic acid (OSA), as compared to the corresponding wild type prenyltransferase; (c) greater than about 1.5-fold rate of formation of 3-geranyl-divarinolinate from geranyl pyrophosphate (GPP) and divarinolic acid as compared to the corresponding wild type prenyltransferase; (d) greater than about 1.5-fold rate of formation of 2-geranyl-olivetol from geranyl pyrophosphate (GPP) and olivetol as compared to the corresponding wild type prenyltransferase; (e) greater than about 1.5-fold rate of formation of 2-geranyl-orcinol from geranyl pyrophosphate (GPP) and orcinol as compared to the corresponding wild type prenyltransferase; (f) greater than about 1.5-fold rate of formation of 2-geranyl-divarinol from geranyl pyrophosphate (GPP) and divarinol as compared to the corresponding wild type prenyltransferase; or any combination of (a)-(f). Even further, in some embodiments, the non-natural prenyltransferase of any of the previous claims enzymatically capable of (a) greater than: about 2-fold, about 5-fold, about 10-fold, about 25-fold, about 50-fold, about 100-fold, about 200-fold, about 300-fold, about 400-fold, or about 500-fold rate of formation of 3-geranyl-olivetolate (3-GOLA), analogs, derivatives, or combinations thereof from geranyl pyrophosphate (GPP) and olivetolic acid (OLA), analogs, derivatives, or combinations thereof as compared to the corresponding wild type prenyltransferase; (b) greater than: about 2-fold, about 5-fold, about 10-fold, about 25-fold, about 50-fold, about 100-fold, about 250-fold, about 300-fold, about 400-fold, about 550-fold, about 2500-fold, about 5000-fold, about 10,000-fold, or about 15,000-fold rate of formation of 3-geranyl-orsellinate (3-GOSA) from geranyl pyrophosphate (GPP) and orsellinic acid (OSA), as compared to the corresponding wild type prenyltransferase; (c) greater than: 2-fold, about 5-fold, about 10-fold, about 25-fold, about 50-fold, about 100-fold or more of formation of 3-geranyl-divarinolinate from geranyl pyrophosphate (GPP) and divarinolic acid as compared to the corresponding wild type prenyltransferase; (d) greater than: 2-fold, about 5-fold, about 10-fold, about 25-fold, about 50-fold, about 100-fold or more of formation of 2-geranyl-olivetol from geranyl pyrophosphate (GPP) and olivetol as compared to the corresponding wild type prenyltransferase; (e) greater than: 2-fold, about 5-fold, about 10-fold, about 25-fold, about 50-fold, about 100-fold, or more of formation of 2-geranyl-orcinol from geranyl pyrophosphate (GPP) and orcinol as compared to the corresponding wild type prenyltransferase; (f) greater than: 2-fold, about 5-fold, about 10-fold, about 25-fold, about 50-fold, about 100-fold, or more of formation of 2-geranyl-divarinol from geranyl pyrophosphate (GPP) and divarinol as compared to the corresponding wild type prenyltransferase; or any combination of (a)-(f).

Some embodiments of the current disclosure are directed to an engineered cell expressing a non-natural prenyltransferase comprising at least one amino acid substitution, two amino acid substitutions, or preferably three or four amino acid substitutions. The cells can be used to promote production of a cannabinoid, such as CBGA (3-GOLA) or cannabigerorcinic acid (CBGOA), or derivatives thereof. Embodiments of the engineered cell may further optionally include one or more additional metabolic pathway transgene(s) to promote improved cannabinoid formation by increasing cannabinoid precursor flux, to generate a cannabinoid derivative, or to improve recovery of the cannabinoid from the engineered cell.

In exemplary embodiments, the engineered cell comprising the non-natural prenyltransferase further comprises an olivetolic acid pathway, such as an olivetolic acid pathway that includes polyketide synthase/olivetol synthase, olivetolic acid cyclase, or both. In exemplary embodiments, the engineered cell further comprises a divarinolic acid (DVA) pathway or an orsellinic acid (OSA) OSA pathway. In exemplary embodiments, the engineered cell comprises a geranyl pyrophosphate (GPP) pathway, such as one that includes geranyl pyrophosphate synthase or geranylgeranyl pyrophosphate synthase. In exemplary embodiments, the GPP pathway comprises a mevalonate (MVA) pathway, a MEP pathway, a non-MEP, non-MVA pathway, or a combination of two or more pathways. In exemplary embodiments, the engineered cell further comprises a cannabinoid synthase.

Other embodiments are directed to compositions including an engineered cell, such as cell culture compositions, and also compositions including one or more product(s) produced from the engineered cell. For example, a composition can include a target cannabinoid product produced by the cells, where the composition has been purified to remove cells or other components useful for cell culturing. For example, the cannabinoid can be cannabigerolic acid (CBGA), tetrahydrocannabivarin (THCV), tetrahydrocannabivarinic acid (THCVA), cannabidivarin (CBDV), cannabidivarinic acid (CBDVA), cannabinol (CBN), cannabinolic acid (CBNA), cannabidiol (CBD), cannabidiolic acid (CBDA), cannabichromene (CBC), cannabichromenic acid (CBCA), cannabigerivarin (CBGV), cannabigerivarinic acid (CBGVA), cannabigerol (CBG), analogs, or derivatives thereof, or combinations thereof. The composition may be treated to enrich or purify the target product or intermediate thereof.

Other embodiments are directed to methods for forming a prenylated aromatic compound. The method includes a step of contacting a substrate comprising a hydrophobic portion and an aromatic substrate with a non-natural prenyltransferase of the disclosure, wherein contacting forms a prenylated aromatic compound. Exemplary aromatic substrates include olivetol, olivetolic acid, divarinol, divarinolic acid, orcinol, and orsellinic acid. The substrate comprising a hydrophobic portion can include an isoprenoid portion, such as dimethylallyl, isopentenyl, geranyl, farnesyl, or geranylgeranyl portions, and can include phosphate groups.

Other embodiments of the disclosure are directed to products made from the target cannabinoid product obtained from methods using the engineered cell. Exemplary products include therapeutic or pharmaceutical compositions, medicinal compositions, systems for in vitro use, diagnostic compositions, and precursor compositions for further chemical modification (e.g. decarboxylation of CBGA to CBG by for example heat or a biocatalyst).

For example, the disclosure can provide purified CBGA, CBG, CBGVA; CBGOA, THCV, THCVA, CBD, CBDA, analogs or derivatives thereof, or combinations thereof, derived from an engineered cell, a cell extract or a cell culture medium of the disclosure, wherein the purified CBGA, CBG, CBGVA; CBGOA, THCV, THCVA, CBD, CBDA, analogs or derivatives thereof, or combinations thereof optionally including PDAL and HTAL at a concentration of no more than about 0.001% to about 0.0001% in the composition.

The disclosure provides compositions wherein the concentration of CBGA, CBG, CBGV, CBGVA; CBGOA, THCV, THCVA, CBD, CBDA, CBDV, CBDVA, CBN, CBNA, CBC, CBCA, analogs or derivatives thereof, or combinations thereof, is at least about 50%, about 55%, about 60%, about 65%, about 70%, about 75%, about 80%, about 85%, about 90%, about 95%, about 97.5%, or about 99% of the total weight of the composition.

Other embodiments of the disclosure are directed to nucleic acids encoding the non-natural prenyltransferases with one or more variant amino acids, as well as expression constructs including the nucleic acids, and engineered cells comprising the nucleic acids or expression constructs.

### DESCRIPTION OF THE DRAWINGS

FIG. 1 shows prenyltransferase-catalyzed reaction of olivetolic acid (OLA) and geranyl diphosphate (GPP) to form the products 3-geranyl-olivetolate (3-GOLA; cannabigerolic acid; CBGA) and 5-geranyl-olivetolate (5-GOLA)

FIG. 2 is diagram of exemplary metabolic pathways showing 3-GOLA formation from hexanoyl-CoA and geranyl diphosphate.

FIG. 3 shows the chemical structures of various examples of aromatic substrate molecules that can be used in a prenyltransferase catalyzed reaction.

FIG. 4 is the amino acid sequence of Streptomyces antibioticus AQJ23_40425 (NCBI Accession number KUN17719.1; 305 amino acids long; SEQ ID NO: 1; NphB).

FIG. 5 shows an alignment of SEQ ID NO: 1 (Streptomyces antibioticus AQJ23_40425) to other prenyltransferase homologs (SEQ ID NOs: 2-15).

FIG. 6A shows reaction of DVA with GPP to form CBGVA, and FIG. 6B shows reaction of OSA with GPP to form CBGOA.

FIG. 7 shows reaction of olivetol with GPP to form CBG (2-GOL).

FIG. 8 is a table of prenyltransferase variant activity for the conversion of OLA+GPP to CBGA and PP. Second generation prenyltransferase variants were prepared by introducing variant amino acids as indicated in the table into template Seq1C (SEQ ID NO: 16, which is SEQ ID NO:1 having Q159S, S212H, and Y286V variant amino acids). Enzyme activity of the second generation prenyl transferase variants is described relative to the activity of template Seq1C, which already provides a 300-fold enzymatic activity increase for conversion of OLA to CBGA over the wild type prenyltransferase enzyme (SEQ ID NO: 1).

FIG. 9 is a table of prenyltransferase variant activity for the conversion of OSA+GPP to CBGOA and PP. Second generation prenyltransferase variants were prepared by introducing variant amino acids as indicated in the table into template Seq1C. Enzyme activity of the second generation prenyltransferase variants is described relative to the activity of template Seq1C, which already provides a >100-fold enzymatic activity increase for conversion of OSA to CBGOA over the wild type prenyltransferase enzyme (SEQ ID NO: 1).

FIG. 10 shows an exemplary olivetolic acid synthesis pathway and exemplary cannabigerolic acid synthesis pathway. The terms tetraketide synthase (TKS) and olivetol synthase (OLS) are used interchangeably.

FIG. 11 shows exemplary pathways of forming geranyl pyrophosphate from isoprenol.

FIG. 12 shows exemplary pathways of forming geranyl pyrophosphate from prenol.

FIG. 13 shows exemplary mevalonate pathway (MVA) and non-mevalonate pathway (MEP). The abbreviations are DXS: 1-Deoxy-D-xylulose 5-phosphate synthase; DXR: 1-Deoxy-D-xylulose 5-phosphate reductoisomerase; CMS: 2-C-methyl-D-erythritol 4-phosphate cytidylyltransferase; CMK: 4-diphosphocytidyl-2-C-methyl-D-erythritol kinase; MECS: 2-C-methyl-D-erythritol 2,4-cyclodiphosphate synthase; HDS: 4-Hydroxy-3-methyl-but-2-enyl pyrophosphate synthase; HDR: 4-Hydroxy-3-methyl-but-2-enyl pyrophosphate reductase; DMAP: Dimethylallyl pyrophosphate; AACT: acetoacetyl-CoA thiolase; HMGS: HMG-CoA synthase; HMGR: HMG-CoA reductase; MVK: mevalonate-3-kinase; PMK: Phosphomevalonate kinase; MVD: mevalonate-5-pyrophosphate decarboxylase; and IDI: isopentenyl pyrophosphate isomerase.

FIG. 14 is the amino acid sequence of a non-natural prenyltransferase triple variant (Seq1C; SEQ ID NO:16; FIG. 14).

### DETAILED DESCRIPTION

The embodiments of the description described herein are not intended to be exhaustive or to limit the disclosure to the precise forms disclosed in the following detailed description. Rather, the embodiments are chosen and described so that others skilled in the art can appreciate and understand the principles and practices of the description.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the disclosure provides non-natural prenyltransferases that can form prenylated alkylbenzenediols or prenylated dihydroxyalkylbenzenoic acids from a substrate comprising a hydrophobic portion such as geranyl pyrophosphate, and alkylbenzenediols or dihydroxyalkylbenzenoic acids, respectively, at increased enzymatic rates as compared to wild-type versions of the enzymes. For example, the disclosure provides non-natural prenyltransferases that are enzymatically capable of greater rates of formation of 3-geranyl-olivetolate (3-GOLA; cannabigerolic acid; CBGA) from geranyl pyrophosphate and olivetolic acid, and/or that are enzymatically capable of greater rates of formation of cannabigerorcinic acid (CBGOA) from orsellinic acid (OSA) and geranyl diphosphate (GPP).

Further, non-natural prenyltransferases of the disclosure with these increased enzymatic rates also demonstrate regioselectivity towards desired products, for example, the variants are capable of regioselectivity (e.g., about 90% or greater, about 95% or greater) to 2-prenylated, 5-alkylbenzene-1,3-diol or 3-prenylated, 2,4-dihydroxy 6-alkylbenzenoic acid from geranyl pyrophosphate and a 5-alkylbenzene-1,3-diol, or a 2,4-dihydroxy 6-alkylbenzenoic acid.

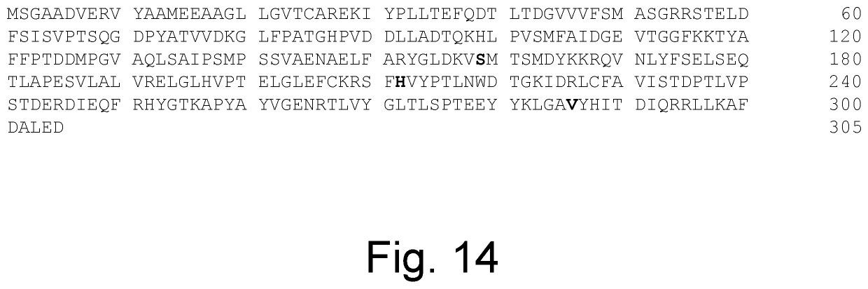

Non-natural prenyltransferase variants of the disclosure include those based on previously identified non-natural prenyltransferase variants already demonstrating improved enzymatic activity and desired regioselectivity over the wild type prenyltransferase. In particular, a non-natural prenyltransferase triple variant (Seq1C; SEQ ID NO:16; FIG. 14) used for generation of further variants is described in commonly assigned International Application No. PCT/US2019/021448 (filed Mar. 8, 2019; Noble, M.), wherein the triple Seq1C variant is based on SEQ ID NO:1 having Q159S, S212H, and Y286V variant amino acids which are shown in bold in FIG. 14. Enzyme activity of the Seq1C triple variant was shown to be >300-fold for conversion of OLA to CBGA, and >100-fold for conversion of OSA to CBGOA over the wild type prenyltransferase enzyme (SEQ ID NO: 1).

Cannabigerolic acid (CBGA; CAS #25555-57-1) has the following chemical names (E)-3-(3,7-dimethyl-2,6-octadienyl)-2,4-dihydroxy-6-pentylbenzoic acid, and 3-[(2E)-3,7-dimethylocta-2,6-dien-1-yl]-2,4-dihydroxy-6-pentylbenzoic acid, and the following chemical structure:

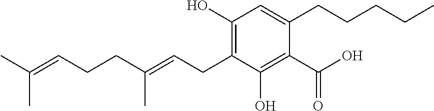 [SMILES: CCCCCc1cc(O)c(C/C=C(\C)CCC=C(C)C)c(O)c1C(=O)O]

CBGA can also be referred to as 3-geranyl-olivetolate (3-GOLA), which reflects the position of the geranyl moiety on the olivetolate moiety.

5-geranyl-olivetolate (5-GOLA) is an enzymatic reaction product of geranyl pyrophosphate and olivetolic acid and has the following structure.

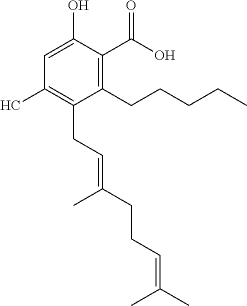 [SMILES: CCCCCc1c(C/C=C(\C)CCC=C(C)C)c(C)cc(O)c1C(=O)O]

As used herein “geranyl-olivetolate” generically refers to either 3-GOLA or 5-GOLA. In an enzymatic reaction using a prenyltransferase variant, “geranyl-olivetolate” products can be produced, although for variants having high regioselectivity to 3-GOLA, very little or trace amounts of 5-GOLA may be produced.

Cannabigerol, the decarboxylated form of 3-GOLA, has the following structure.

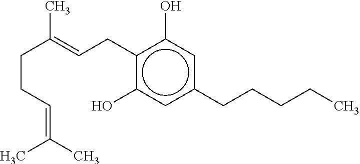

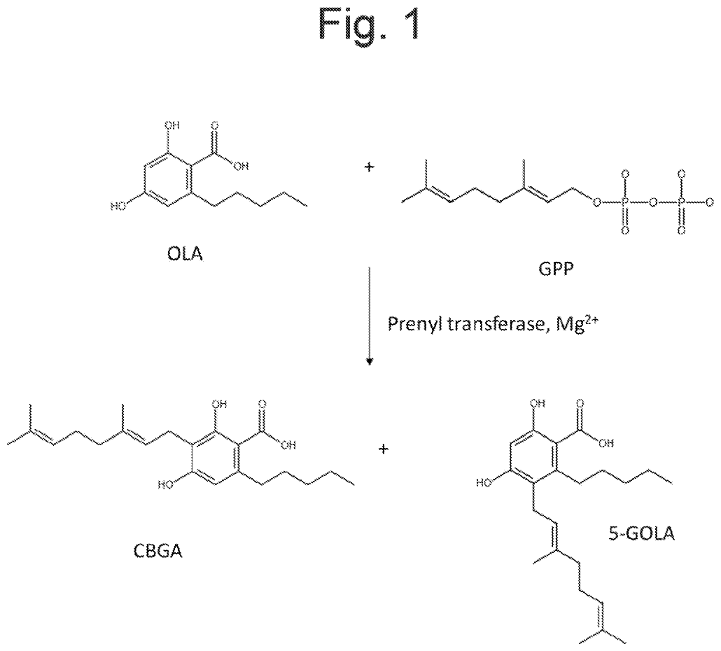

Cannabigerol (CBG; 2-GOL; 2-[(2E)-3,7-dimethylocta-2,6-dienyl]-5-pentylbenzene-1,3-diol; CAS #: 25654-31-3) can be considered a “derivative” of GOLA/CBGA. 4-GOL is the isomer of 2-GOL prenylated at the 4 position on the aromatic ring (i.e., between a hydroxyl group at the 1 or 3 position on the aromatic ring and the pentyl group). CBG can be formed by decarboxylation of CBGA, for example by heat or by catalysis, which can be a biocatalyst such as an enzyme, whole cell, or cell extract. In addition to the use of olivetolic acid (OLA) for forming 3-GOLA/CBGA by reaction as catalyzed by prenyltranferase (see FIG. 1), the disclosure also contemplates the use of other substrate molecules as a replacement to OLA.

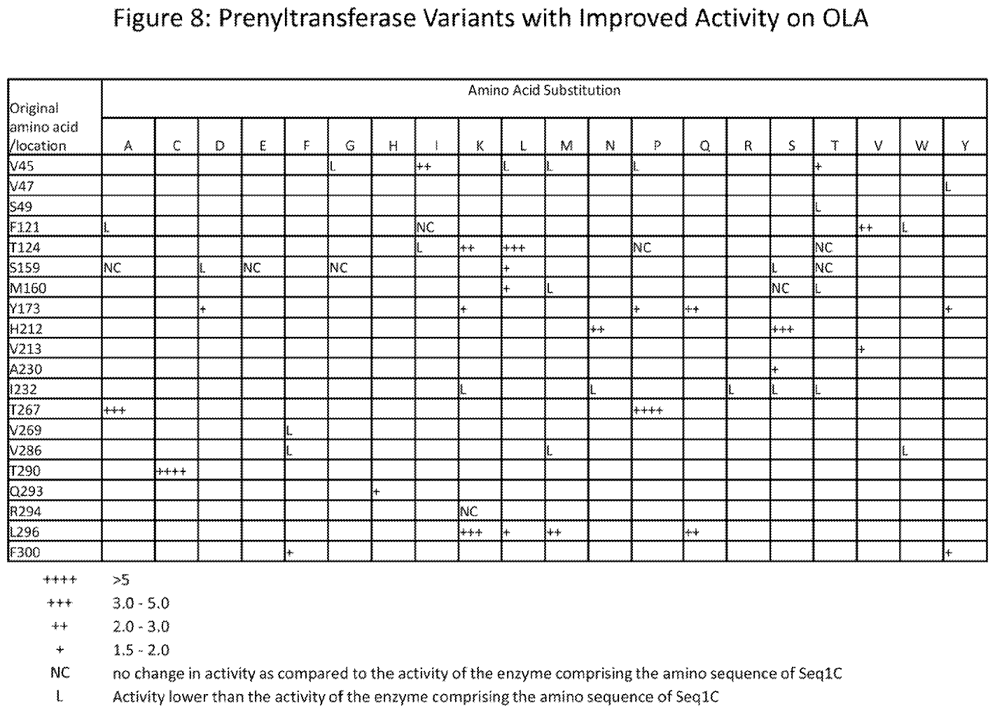

Cannabigerol (CBG; 2-GOL) can also be regioselectively formed (i.e., over formation of 4-GOL) from olivetol and geranyl pyrophosphate (see FIG. 8) using non-natural prenyltransferase variants of the disclosure.

Cannabigerovarinic acid (CBGVA; 3-GDVA; 3-[(2E)-3,7-dimethylocta-2,6-dienyl]-2,4-dihydroxy-6-propylbenzoic acid; C₂₀H₂₈O₄; #64924-07-8) is a minor cannabinoid.

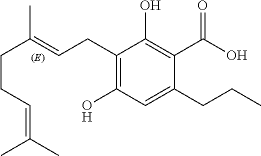 [SMILES: CCCc1cc(O)c(C/C=C(\C)CCC=C(C)C)c(O)c1C(=O)O]

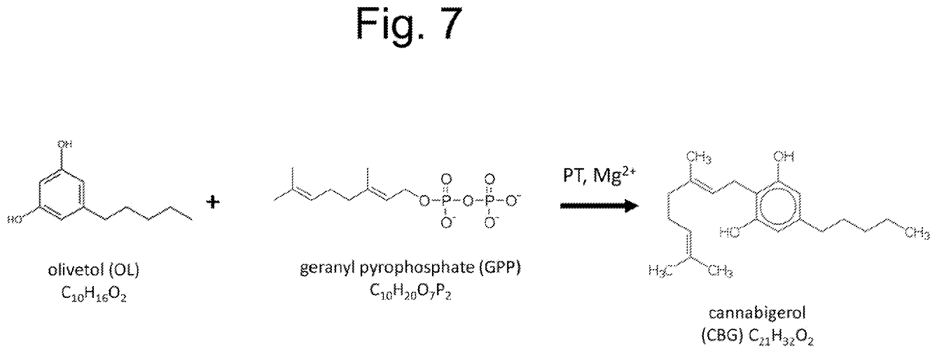

5-GDVA is the isomeric form with the (2E)-3,7-dimethylocta-2,6-dienyl group attached to the 5 position on the aromatic ring. FIG. 7A shows reaction of divarinolic acid (DVA) and geranyl diphosphate (GPP) to form the product cannabigerovarinic acid (CBGVA).

Cannabigerorcinic acid (CBGOA; 3-GOSA; 3-[(2E)-3,7-dimethyl-2,6-octadien-1-yl]-2,4-dihydroxy-6-methyl-benzoic acid; C₁₈H₂₄O₄; #69734-83-4) is another minor cannabinoid.

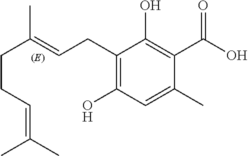 [SMILES: CC(C)=CCC/C(C)=C/Cc1c(O)cc(C)c(C(=O)O)c1O]

5-GOSA is the isomeric form with the (2E)-3,7-dimethyl-2,6-octadien-1-yl group attached to the 5 position on the aromatic ring. FIG. 7B shows reaction of orsellinic acid (OSA) and geranyl diphosphate (GPP) to form the product cannabigerorcinic acid (CBGOA).

The term “regioselective” and “regioselectivity” as used in a “regioselective reaction” refers to a direction of bond making or breaking that occurs preferentially over all other possible directions. A reaction between substrate A and substrate B may yield two or more reaction products (e.g., product C, product D, etc.) Regioselectivity can be understood by determining the relative molar amount of products formed. For example, in an enzymatic reaction wherein substrate A and substrate B react to form a product mixture of product C and product D, and wherein the molar ratio of product C:product D is greater than 1:1, respectively, in the product mixture, the reaction is regioselective to product C. Wherein the molar ratio of product C:product D is 9:1 or greater, respectively, in the product mixture, in the product mixture, the reaction has about 90% or greater regioselectivity to product C.

The disclosure also contemplates methods for, generally, forming a prenylated aromatic compound. The method involves contacting a substrate comprising a hydrophobic portion and an aromatic substrate with a non-natural prenyltransferase of the disclosure to form a prenylated aromatic compound. For example, in particular, the disclosure contemplates use of various aromatic substrates such as olivetol, olivetolic acid, divarinol, divarinolic acid, orcinol, and orsellinic acid in such a prenyltransferase-catalyzed reaction. The substrate comprising a hydrophobic portion can include an isoprenoid portion, a geranyl portion, a farnesyl portions, and one or more phosphate groups. Exemplary hydrophobic portions include partially saturated hydrocarbon moieties having an amount of carbons in the range of 5-20. The method can be performed in vivo (e.g., within the engineered cell) or in vitro. Exemplary substrates comprising a hydrophobic portion include, but are not limited to, dimethylallyl pyrophosphate (DMAPP; C5), geranyl pyrophosphate (GPP; C10), farnesyl pyrophosphate (FPP; C15), and geranylgeranyl pyrophosphate (GGPP; C20).

Also described are engineered cells expressing a non-natural prenyltransferase, optionally including one or more additional metabolic pathway transgene(s); cell culture compositions including the cells; methods for promoting production of the target cannabinoid or derivative thereof from the cells; compositions including the target cannabinoid or derivative; and products made from the target product or intermediate.

The term “non-naturally occurring”, when used in reference to an organism (e.g., microbial) is intended to mean that the organism has at least one genetic alteration not normally found in a naturally occurring organism of the referenced species. Naturally-occurring organisms can be referred to as “wild-type” such as wild type strains of the referenced species. Likewise, a “non-natural” polypeptide or nucleic acid can include at least one genetic alteration not normally found in a naturally-occurring polypeptide or nucleic acid. Naturally-occurring organisms, nucleic acids, and polypeptides can be referred to as “wild-type” or “original” such as wild type strains of the referenced species. Likewise, amino acids found in polypeptides of the wild type organism can be referred to as “original” with regards to any amino acid position.

The term “about” which can be used to modify, for example, a value relating to an amount, such as a concentration, a volume, a percent identity, and like values, and ranges thereof, employed in describing the embodiments of the disclosure, refers to variation in the numerical quantity that can occur, for example, through typical measuring and handling procedures, through inadvertent error in these procedures; through differences in the manufacture, source, or purity of starting materials or ingredients used to carry out the methods, and like proximate considerations, with the value being within 5%, or less, of the numerical quantity specified.

A genetic alteration that makes an organism non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

For example, in some modes of practice, in order to provide a soluble aromatic prenyltransferase variant of the disclosure, a soluble ABBA type prenyltransferase from Streptomyces antibioticus AQJ23_40425 (NCBI Accession number KUN17719.1; 305 amino acids long; SEQ ID NO: 1), can be selected as a template. Variants, as described herein, can be created by introducing into the SEQ ID NO: 1 template or homolog there (SEQ ID NOs: 2-15) one or more amino acid substitutions as described herein to increased activity to a desired product, such as CBGA (3-GOLA). In other modes of practice, in order to provide a soluble aromatic prenyltransferase variant of the disclosure, a previously formed variant having improved activity and/or regioselectivity, such as a single, double, or triple variant (e.g., the Seq1C variant, which is represented by SEQ ID NO:16, is based on SEQ ID NO:1 and has Q159S, S212H, and Y286V variant amino acids, or a homolog of SEQ ID NO:1 such as SEQ ID NOs: 2-15 such as a single, double, or triple variant) can be used as a template and then further one or more amino acid substitutions as described herein can be introduced to further increase activity to a desired product, such as CBGA (3-GOLA).

If a previously formed or identified variant, such as SEQ ID NO: 16 which has variant amino acids at positions 159, 212, and 286 and is derived from SEQ ID NO:1, is further modified to include one or more other amino acid variations of the disclosure, such as further modified variant may be referred to herein as a “second generation variant” if desired. SEQ ID NO: 16 already has improved activity and regioselectivity over the wild type enzyme (as described International Application No. PCT/US2019/021448), and therefore a second generation variant can provide further improved activity and/or a further beneficial property or properties as compared to the first generation variant.

In some cases, a “homolog” of the prenyltransferase SEQ ID NO: 1, is first identified. A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

Genes sharing a desired amount of identify (e.g., 45%, 50%, 55%, or 60% or greater) to the Streptomyces antibioticus AQJ23_4042 prenyltransferase SEQ ID NO: 1, including homologs, orthologs, and paralogs, can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor.

Computational approaches to sequence alignment and determination of sequence identity include global alignments and local alignments. Global alignment uses global optimization to forces alignment to span the entire length of all query sequences. Local alignments, by contrast, identify regions of similarity within long sequences that are often widely divergent overall. For understanding the identity of a target sequence to the Streptomyces antibioticus s AQJ23_4042 SEQ ID NO: 1 prenyltransferase template a global alignment can be used. Optionally, amino terminal and/or carboxy-terminal sequences of the target sequence that share little or no identity with the template sequence can be excluded for a global alignment and generation of an identity score.

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%).

Pairwise global sequence alignment can be carried out using Streptomyces antibioticus AQJ23_4042 prenyltransferase SEQ ID NO: 1 as the template. Alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (http://balign.sourceforge.net/). Default parameters are used for the alignment and BLOSUM62 was used as the scoring matrix.

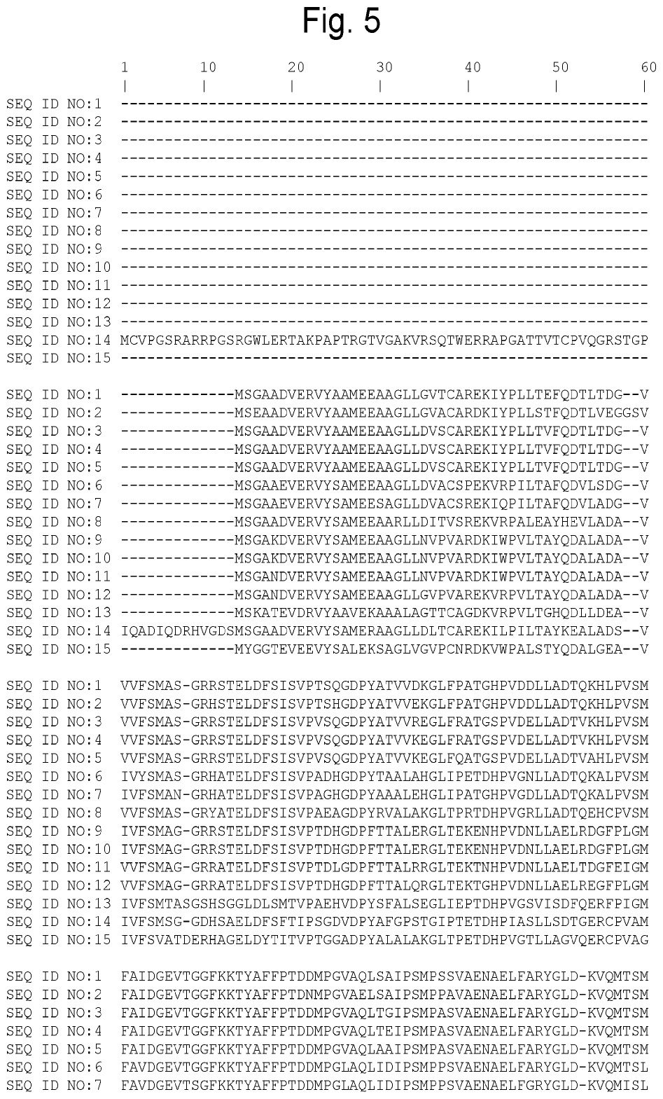

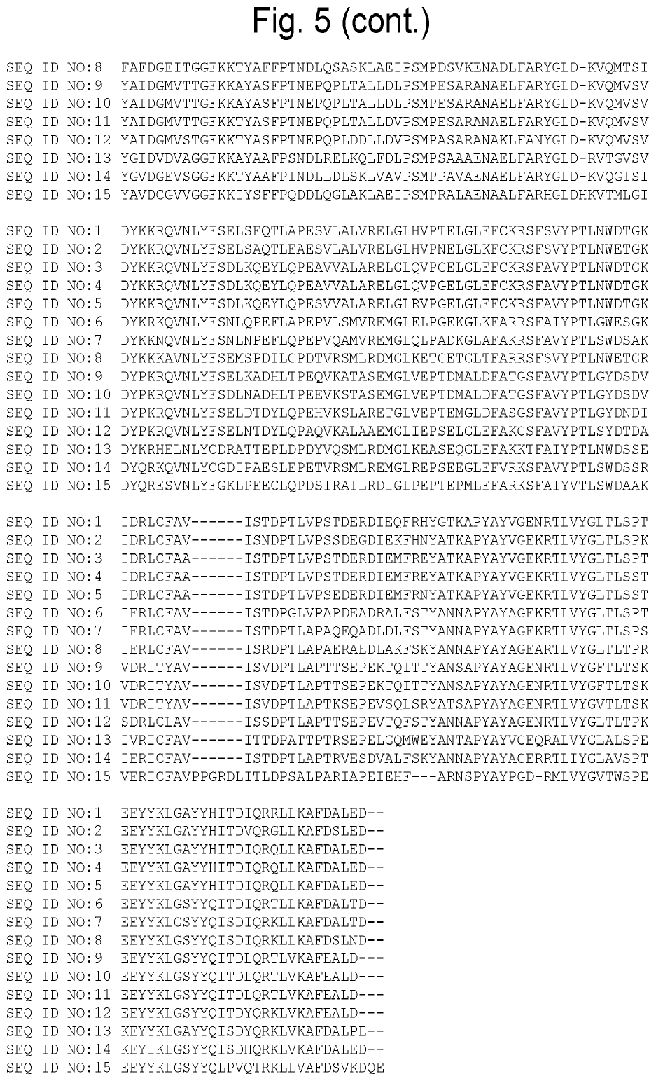

For the purpose of amino acid position numbering, SEQ ID NO: 1 is used as the reference sequence. For example, mention of amino acid position 49 is in reference to SEQ ID NO:1, but in the context of a different prenyltransferase sequence (a target sequence or other template sequence) the corresponding amino acid position for variant creation may have the same or different position number, (e.g. 48, 49 or 50). In some cases, the original amino acid and its position on the SEQ ID NO: 1 reference template will precisely correlate with the original amino acid and position on the target prenyltransferase. In other cases, the original amino acid and its position on the SEQ ID NO: 1 template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the template position. In other cases the original amino acid on the SEQ ID NO: 1 template will not precisely correlate with the original amino acid on the target. However, one can understand what the corresponding amino acid on the target sequence is based on the general location of the amino acid on the template and the sequence of amino acids in the vicinity of the target amino acid, especially referring to the alignment provided in FIG. 5. It is understood that additional alignments can be generated with prenyltransferase sequences not specifically disclosed herein, and such alignments can be used to understand and generate new prenyltransferase variants in view of the current disclosure. In some modes of practice, the alignments can allow one to understand common or similar amino acids in the vicinity of the target amino acid, and those amino acids may be viewed as “sequence motif” having a certain amount of identity or similarity to between the template and target sequences. Those sequence motifs can be used to describe portions of prenyltransferase sequences where variant amino acids are located, and the type of variation(s) that can be present in the motif.

In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2.0.8 (Jan. 5 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off Those skilled in the art will know what modifications can be made to the above parameters to either increase or decrease the stringency of the comparison, for example, and determine the relatedness of two or more sequences.

FIG. 5 shows an alignment of SEQ ID NO: 1 (Streptomyces antibioticus AQJ23_40425) to other prenyltransferase homologs (SEQ ID NOs 2-15). As described in International Application No. PCT/US2019/021448, these homologs were found by BLAST search, and range in sequence identity to SEQ ID NO: 1 from 88.9%-50.8% (SEQ ID NOs 2-15). Also described in International Application No. PCT/US2019/021448, these homologs were tested for activity on OLA and GPP in cell lysate. Low, but measurable, activity was identified in all 15 homologs, with SEQ ID NO: 1 among the highest observed. The low activities of wild-type homologs observed are in accord with that reported by Kumano, as previously mentioned.

In some embodiments, a prenyltransferase template into which the one or more variations (also referred to herein as mutation or substitution) are introduced to create a variant that is a prenyltransferase sequence having about 50% or greater identity, about 60% or greater identity, about 65% or greater identity, about 70% or greater identity, about 75% or greater identity, about 80% or greater identity, about 85% or greater identity, about 87.5% or greater identity, about 90% or greater identity, about 92.5% or greater identity, about 95% or greater identity, about 98% or greater identity, about 99% or greater identity, or 100% identity to any of SEQ ID NOs: 1-16, with the one or more variations that are introduced into the template selected from the group consisting of: selected from a variation at position 45; 121, wherein the amino acid variation is 121V; 124, wherein the amino acid variation is selected from 124K and 124L; 160; 173; 212, wherein the amino acid variation is 212N; 232, wherein the amino acid variation is selected from 232K, 232N, 232R, and 232S; 267, wherein the amino acid variation is selected from 267A and 267P; 269, wherein the amino acid variation is 269F; 286, wherein the amino acid variation is 286W; 290; 294; 296, wherein the amino acid variation is selected from 296K, 296M, and 296Q; 300, the amino acid locations relative to SEQ ID NO:1 or a corresponding amino acid location in any of SEQ ID NOs: 2-16, the variant having prenyltransferase activity.

The variants of this embodiment can be “single substitution” variants, with the template sequence changed by only one amino acid of the described group (i.e., 45G, 45I, etc.), or variants of this embodiment can be “multiple substitution” variants, with the template sequence changed by one or more amino acid(s) of the described group (i.e., 45G, 45I, etc.), and optionally one or more other amino acids outside of the described group. In some preferred embodiments, the non-natural prenyltransferase variant is based on a template having 100% identity or less than 100% identity of one of SEQ ID NOs: 1-16, and having a single variant amino acid selected from the group consisting of: 45I; 45T; 45S, 121V; 124K; 124L; 160L; 160V; 160I; 173D; 173K; 173P; 173Q; 173E; 173F; 212N; 232K; 232N; 232R; 232S; 267A; 267P; 269F; 286W; 290A; 290I; 290M; 290S; 294K; 294H; 296K; 296M; 296Q; and 300Y. In other preferred embodiments, the non-natural prenyltransferase variant is based on a template having 100% identity or less than 100% identity of one of SEQ ID NOs: 1-16, and having one variant amino acid selected from the group consisting of: 45I; 45T; 45S, 121V; 124K; 124L; 160L; 160V; 160I; 173D; 173K; 173P; 173Q; 173E; 173F; 212N; 232K; 232N; 232R; 232S; 267A; 267P; 269F; 286W; 290A; 290I; 290M; 290S; 294K; 294H; 296K; 296M; 296Q; and 300Y, and one or more other variant amino acids outside of this group.

In some embodiments, a prenyltransferase template into which the one or more variations are introduced to create a variant is a prenyltransferase sequence having about 50% or greater identity, about 60% or greater identity, about 65% or greater identity, about 70% or greater identity, about 75% or greater identity, about 80% or greater identity, about 85% or greater identity, about 87.5% or greater identity, about 90% or greater identity, about 92.5% or greater identity, about 95% or greater identity, about 98% or greater identity, about 99% or greater identity, or 100% identity to any of SEQ ID NOs: 1-15, and having at least three amino acid variations as compared to a wild type template, wherein at least two of the amino acid variations are selected from variations at locations 159, 212, and 286, and at least one other amino acid variation is selected from a variation at position 45, 47, 45, 47, 49, 121, 124, 160, 173, 213, 230, 232, 267, 269, 290, 294, 296, and 300, the amino acid locations relative to SEQ ID NO:1 or a corresponding amino acid location in any of SEQ ID NOs:2-15, the variant having prenyltransferase activity.

For example, in embodiments, the non-natural prenyltransferase has about 50% or greater identity to any one of SEQ ID NOs:1-15, and can include at least two amino acid variations selected from Q159S, S212H, and Y286V, such as a prenyltransferase having Q159S and Y286V variations, or all three Q159S, S212H, and Y286V variations, and at least one other amino acid variation at a position(s) selected from 45, 47, 49, 121, 124, 160, 173, 213, 230, 232, 267, 269, 290, 294, 296, and 300, as described herein. For example, the at least one other amino acid variation can be selected from 45I; 45T; 45S, 121V; 124K; 124L; 160L; 160V; 160I; 173D; 173K; 173P; 173Q; 173E; 173F; 212N; 232K; 232N; 232R; 232S; 267A; 267P; 269F; 286W; 290A; 290I; 290M; 290S; 294K; 294H; 296K; 296M; 296Q; and 300Y.

Preferably, the one or more amino acid variation(s) is selected from the group consisting of: 45I; 45T; 121V; 124K; 124L; 160L; 173D, 173K, 173P, 173Q; 212N; 232K; 232N; 232R; 232S; 267A; 267P; 269F; 286W; 294K; 296K; 296M; 296Q; and 300Y.

For example, a non-natural prenyltransferase of the disclosure can have four, five, six, seven, eight, nine, ten, eleven, twelve, thirteen, fourteen, fifteen, sixteen, seventeen, eighteen, nineteen, twenty, or more amino acid variations as compared to a wild type prenyltransferase.

Exemplary non-natural prenyltransferases of the disclosure that can have at least three amino acid variations (i.e., having three amino acid variations, or more than three amino acid variations) at the following positions relative to SEQ ID NO:1 or a corresponding amino acid location in any of SEQ ID NOs:2-15, or in a template having 50% or greater identity to any one of SEQ ID NOs:1-15, include those wherein the variations are selected from: (i) 45I, (ii) 159S, (iii) and 286V; (i) 45T, (ii) 159S, and (iii) 286V; (i) 121V, (ii) 159S, and (iii) 286V; (i) 124K, (ii) 159S, and (iii) 286V; (i) 124L, (ii) 159S, and (iii) 286V; (i) 159S, (ii) 160L and (iii) 286V; (i) 159S, (ii) 160L and (iii) 286V; (i) 159S, (ii) 160S and (iii) 286V; (i) 159S, (ii) 173D and (iii) 286V; (i) 159S, (ii) 173K and (iii) 286V; (i) 159S, (ii) 173P and (iii) 286V; (i) 159S, (ii) 173Q and (iii) 286V; (i) 159S, (ii) 173Y and (iii) 286V; (i) 159S, (ii) 212H and (iii) 286V; (i) 159S, (ii) 230S and (iii) 286V; (i) 159S, (ii) 267P and (iii) 286V; i) 159S, (ii) 286V; and (iii) 293H; (i) 159S, (ii) 286V; and (iii) 294K; (i) 159S, (ii) 286V; and (iii) 296K; (i) 159S, (ii) 286V; and (iii) 296L; (i) 159S, (ii) 286V; and (iii) 296M; (i) 159S, (ii) 286V; and (iii) 296Q; (i) 159S, (ii) 286V; and (iii) 296M; (i) 159S, (ii) 286V; and (iii) 300F; and (i) 159S, (ii) 286V; and (iii) 300Y.

Exemplary non-natural prenyltransferases of the disclosure that can have at least four amino acid variations (i.e., having four amino acid variations, or more than four amino acid variations) at the following positions relative to SEQ ID NO:1 or a corresponding amino acid location in any of SEQ ID NOs:2-15, or in a template 50% or greater identity, about 60% or greater identity, about 65% or greater identity, about 70% or greater identity, about 75% or greater identity, about 80% or greater identity, about 85% or greater identity, about 87.5% or greater identity, about 90% or greater identity, about 92.5% or greater identity, about 95% or greater identity, about 98% or greater identity, about 99% or greater identity, or 100% identity to any of SEQ ID NOs: 1-15, include those wherein the variations are selected from: (i) 45I, (ii) 159S, (iii) 212H, and (iv) 286V; (i) V45T, (ii) 159S, (iii) 212H, and (iv) 286V; (i) 121V, (ii) 159S, (iii) 212H, and (iv) 286V; (i) 124K, (ii) 159S, (iii) 212H, and (iv) 286V; (i) 124L, (ii) 159S, (iii) 212H, and (iv) 286V; (i) 159S, (ii) 160L, (iii) 212H, and (iv) 286V; (i) 159S, (ii) 160L, (iii) 212H, and (iv) 286V; (i) 159S, (ii) 160S, (iii) 212H, and (iv) 286V; (i) 159S, (ii) 173D, (iii) 212H, and (iv) 286V; (i) 159S, (ii) 173K, (iii) 212H, and (iv) 286V; (i) 159S, (ii) 173P, (iii) 212H, and (iv) 286V; (i) 159S, (ii) 173Q, (iii) 212H, and (iv) 286V; (i) 159S, (ii) 173Y, (iii) 212H, and (iv) 286V; (i) 159S, (ii) 212H, (iii) 213V, and (iv) 286V; (i) 159S, (ii) 212H, (iii) 230S, and (iv) 286V; (i) 159S, (ii) 212H, (iii) 267P, and (iv) 286V; i) 159S, (ii) 212H, (iii) 286V, and (iv) 293H; (i) 159S, (ii) 212H, (iii) 286V, and (iv) 294K; (i) 159S, (ii) 212H, (iii) 286V, and (iv) 296K; (i) 159S, (ii) 212H, (iii) 286V, and (iv) 296L; (i) 159S, (ii) 212H, (iii) 286V, and (iv) 296M; (i) 159S, (ii) 212H, (iii) 286V, and (iv) 296Q; (i) 159S, (ii) 212H, (iii) 286V, and (iv) 296M; (i) 159S, (ii) 212H, (iii) 286V, and (iv) 300F; and (i) 159S, (ii) 212H, (iii) 286V, and (iv) 300Y, the variant having prenyltransferase activity.

In some embodiments, the non-natural prenyltransferase further comprises one or more other amino acid variation(s) not specified herein, and selected from a variation at one or more position(s) selected from the group consisting of 45, 47, 49, 121, 124, 160, 173, 213, 230, 232, 267, 269, 290, 294, 296, and 300. In some embodiments, the non-natural prenyltransferase further comprises one or more other amino acid variation(s) and selected from a variation at one or more position(s) selected from the group consisting 124S, 232T, 296I, 300P; and 300I.

In some embodiments, in addition to the one or more variant amino acids of the disclosure, the non-natural prenyltransferase can have a further amino acid variation at one or more of positions 121, 161, 175, 211, 214, 230, 268, 269, 284, 285, 286, 288, 293, 295. For example, the non-natural prenyltransferase can further comprise one or more amino acid variation(s) selected from the group consisting of: 47S, 47N, 47G; 121L; 161R, 161H, 161S; 175H, 175K, 175R; 211H; 211N; 214H; 230S; 268Y; 269N; 284S; 285Y; 286F, 286L, 286M, 286P, 286T, 286V, 286I, 286A; 288V, 288I; 296I; 293H, 293M, 293F, 293W, 293C, 293C, 293A, 293S, 293V, 293D, 293Y, 293E, 293I, and 293T. In some embodiments, in addition to the one or more variant amino acids of the disclosure, the non-natural prenyltransferase can have a further amino acid variation that is selected from 47S, 47N, 47G; 211H; 211N; 269N; 284S; and 296I as described by Valliere et al. (Nature Communications, 2019, 10:565).

One, or more than one, amino acid variation can be described relative to the location of a particular amino acid in a wild type prenyltransferase template sequence. Identification of locations in the template that when substituted with variant amino acids which provide desired activity and regioselectivity can be determined by testing methods as described herein.

For example, in the prenyltransferase template SEQ ID NO: 1 one or more of the following positions may be subject to substitution with an amino acid that is different than the wild type amino acid at that location: V45, V47, S49, F121, T124, Q159, M160, Y173, V213, A230, I232, T267, V269, T290, Q293, R294, L296, and F300.

However, in other prenyltransferase templates, the location of the target amino acid for substitution may be different but corresponds to the positions identified for SEQ ID NO. 1, which is the reference template herein. For example, in a prenyltransferase sequence that is different than SEQ ID NO: 1, the target amino acids can be shifted in the range of 10 to −1, or in the range of +1 to +10, based on the particular amino acid variation location. For example, using the alignment of FIG. 5 as a guide, amino acid position 159 of SEQ ID NO: 1 corresponds to position 161 in SEQ ID NO:2, and amino acid position 293 of SEQ ID NO: 1 corresponds to position 295 in SEQ ID NO:2. In some cases, the shift can vary along the length of the sequence that is aligned to SEQ ID NO:1. For example, the shift may increase or decrease after a first stretch of amino acids in the aligned sequence, an then may increase or decrease after a second stretch of amino acids in the aligned sequence, etc. The shift of shifts can be determined by the gaps between the template and aligned sequence along the length of the proteins.

Art-known methods can be used for the testing the enzymatic activity of prenyltransferase, and such methods can be used to test activity of prenyltransferase variant enzymes as well. As a general matter, an in vitro reaction composition including a prenyltransferase variant (purified or in cell lysate or cell extract), geranyl pyrophosphate and olivetolic acid (substrates) can convert the substrates to the product geranyl-olivetolate (e.g., GOLA). Of particular interest herein is conversion of geranyl pyrophosphate and olivetolic acid to CBGA. See the attached figures.

In some embodiments, non-natural prenyltransferases with one or more variant amino acids as describe herein are enzymatically capable of a greater rate of formation of cannabigerolic acid from geranyl pyrophosphate and olivetolic acid, as compared to the wild type prenyltransferase. Variants were also identified that displayed very high activity on the order of about 300-fold or greater rate of formation of cannabigerolic acid from geranyl pyrophosphate and olivetolic acid, as compared to the wild type prenyltransferase. For example, the increase in rate of formation of cannabigerolic acid from geranyl pyrophosphate and olivetolic acid, as compared to the wild type prenyltransferase, can be in the range of about 1.5× to about 750×, about 5× to about 750×, or about 10× to about 750× as determined in an in vitro enzymatic reaction using purified prenyltransferase variant.

For example, non-natural prenyltransferases of the disclosure are enzymatically capable of greater than: about 1.5-fold, about 2-fold, about 5-fold, about 10-fold, about 25-fold, about 50-fold, about 100-fold, about 200-fold, about 300-fold, about 400-fold, or about 500-fold of formation of 3-geranyl-olivetolate (3-GOLA), analogs, derivatives, or combinations thereof from geranyl pyrophosphate (GPP) and olivetolic acid (OLA), analogs, derivatives, or combinations thereof, as compared to the corresponding wild type prenyltransferase.

Non-natural prenyltransferases of the disclosure can also be enzymatically capable of greater than: about 1.5-fold, about 2-fold, about 5-fold, about 10-fold, about 25-fold, about 50-fold, about 100-fold, about 250-fold, about 300-fold, about 400-fold, about 550-fold, about 2500-fold, about 5000-fold, about 10,000-fold, or about 15,000-fold rate of formation of 3-geranyl-orsellinate (3-GOSA from geranyl pyrophosphate (GPP) and orsellinic acid (OSA), as compared to the corresponding wild type prenyltransferase;

Non-natural prenyltransferases of the disclosure can also be enzymatically capable of greater than: about 1.5-fold, about 2-fold, about 5-fold, about 10-fold, about 25-fold, about 50-fold, or about 100-fold rate of formation of 3-geranyl-divarinolinate from geranyl pyrophosphate (GPP) and divarinolic acid as compared to the corresponding wild type prenyltransferase

Non-natural prenyltransferases of the disclosure can also be enzymatically capable of greater than: about 1.5-fold, about 2-fold, about 5-fold, about 10-fold, about 25-fold, about 50-fold, or about 100-fold rate of formation of 2-geranyl-olivetol from geranyl pyrophosphate (GPP) and olivetol as compared to the corresponding wild type prenyltransferase.

Non-natural prenyltransferases of the disclosure can also be enzymatically capable of greater than: about 1.5-fold, about 2-fold, about 5-fold, about 10-fold, about 25-fold, about 50-fold, or about 100-fold rate of formation of 2-geranyl-orcinol from geranyl pyrophosphate (GPP) and orcinol as compared to the corresponding wild type prenyltransferase;

Non-natural prenyltransferases of the disclosure can also be enzymatically capable of greater than: about 1.5-fold, about 2-fold, about 5-fold, about 10-fold, about 25-fold, about 50-fold, or about 100-fold rate of formation of 2-geranyl-divarinol from geranyl pyrophosphate (GPP) and divarinol as compared to the corresponding wild type prenyltransferase.

Non-natural prenyltransferases of the disclosure can also display increased enzymatic activity for converting one or more of the aforementioned substrates to corresponding products.

The increase of enzymatic activity can be seen in in vitro reactions using cell lysates from bacteria expressing the prenyltransferase variants, or from purified preparations of the prenyltransferase variants (e.g., purified from cell lysates). As noted in International Application No. PCT/US2019/021448, it was observed that for many variants, purified preparations showed increased enzymatic activity over the cell lysates, indicating that in some cell lysates prenyltransferase may suffer from insolubility or other event that reduces enzyme activity. As such, a purified prenyltransferase preparation may show about 1.5-fold, about 2-fold, about 5-fold, about 10-fold, or even about 20-fold improvement of enzyme activity over the corresponding cell lysate, controlling for equal amounts of the prenyltransferase in the enzymatic assay.

Using a purified prenyltransferase preparation the rate of formation of CBGA can be determined. The rate can be expressed in terms of μM CBGA/min/μM enzyme. Reaction conditions can be as follows: 50 mM HEPES, pH 7.5 buffer containing 1 mM geranyl pyrophosphate (Sigma-Aldrich) and 1 mM olivetolic acid (Santa Cruz Biotechnology) and 5 mM magnesium chloride. Reactions are initiated by addition of purified prenyltransferase and then incubated for a measured period of 0.5 to 2 hours, quenched with acetonitrile to a final concentration of 65%, then centrifuged to pellet denatured protein. Supernatants are transferred to 96-well plates for LCMS LCMS analysis of CBGA (3-GOLA) and 5-GOLA.

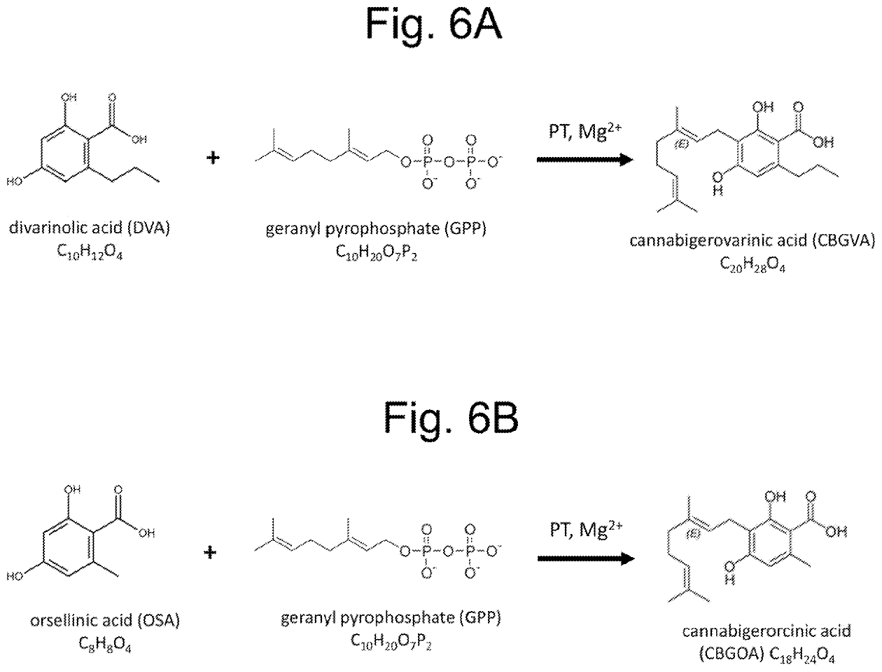

Likewise, using a purified prenyltransferase variant preparation, the rate of formation of cannabigerovarinic acid (CBGVA) from geranyl pyrophosphate and divarinolic acid (DVA) can be determined using similar methods, as well as the rate of formation of cannabigerorcinic acid (CBGOA) from geranyl pyrophosphate and orsellinic acid (OSA) (see FIGS. 6A and 6B) and the rate of formation of cannabigerol (CBG) from olivetol and geranyl pyrophosphate (see FIG. 7).

In embodiments, the prenyltransferase variants provide a rate of formation of CBGA of greater than 0.005 μM CBGA/min/μM enzyme, greater than about 0.010 μM CBGA/min/μM enzyme, greater than about 0.020 μM CBGA/min/μM enzyme, greater than about 0.050 μM CBGA/min/μM enzyme, greater than about 0.100 μM CBGA/min/μM enzyme, greater than about 0.250 μM CBGA/min/μM enzyme, greater than about 0.500 μM CBGA/min/μM enzyme, such as in the range of about 0.005 μM or 0.010 μM to about 1.250 μM CBGA/min/μM enzyme, or in the range of about 0.020 μM to about 1.0 μM CBGA/min/μM enzyme.

In embodiments, the prenyltransferase variants provide a rate of formation of CBGVA from DVA and GPP, of CBGOA from OSA and GPP, or of CBG from olivetol and GPP, according to any of the rates as described herein.

In some embodiments, non-natural prenyltransferases with one or more variant amino acids as describe herein are enzymatically capable of providing regioselectivity to 3-geranyl-olivetolate (CBGA; 3-GOLA). In some embodiments the non-natural prenyltransferases with one or more variant amino acids providing an amount of regioselectivity to 3-geranyl-olivetolate CBGA of about 60% or greater, about 70% or greater, about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.2% or greater, about 99.4% or greater, about 99.5% or greater, about 99.6% or greater, about 99.7% or greater, about 99.8% or greater, about 99.9% or greater, about 99.95% or greater, or 100% regioselectivity to 3-geranyl-olivetolate (CBGA; 3-GOLA) of the total geranyl olivetolate (3-GOLA plus 5-GOLA) as determined in an in vitro enzymatic reaction using purified prenyltransferase variant. Accordingly, of the geranyl-olivetolate reaction products, 5-GOLA is in an amount of less than about 10% (wt), less than about 9% (wt), less than about 8% (wt), less than about 7% (wt), less than about 6 (wt), less than about 5% (wt), less than about 4% (wt), less than about 3% (wt), less than about 2% (wt), less than about 1% (wt), less than about 0.8% (wt), less than about 0.6% (wt), less than about 0.5% (wt), less than about 0.4% (wt), less than about 0.3% (wt), less than about 0.2% (wt), less than about 0.1% (wt), less than about 0.05% (wt) or 0.0% (wt). In view of the improved regioselectivity of the prenyltransferase variants, the disclosure also provides compositions that are enriched for desired cannabinoids and derivatives thereof. In particular, the disclosure provides compositions enriched for CBGA (3-GOLA) and/or CBG. Enriched compositions include those that are pharmaceutical compositions as well as those that are used for non-pharmaceutical purposes, such as having 90% or greater 3-GOLA as described herein, or other desired derivatives depending on the provided substrate (e.g. olivetol, olivetolic acid, et.) as described elsewhere herein. In some embodiments, non-natural prenyltransferase with one or more variant amino acids as describe herein display an increase in rate of formation of cannabigerolic acid from geranyl pyrophosphate and olivetolic acid, in any of the amounts described herein, and regioselectivity in any of the amounts as described herein.

In some embodiments, non-natural prenyltransferases with one or more variant amino acids as describe herein are enzymatically capable of providing regioselectivity to 3-geranyl-orsellinate (3-GOSA), an isomer of cannabigerorcinic acid (CBGOA) formed after reacting GPP and OSA. In some embodiments the non-natural prenyltransferases with one or more variant amino acids providing an amount of regioselectivity to 3-GOSA of about 60% or greater, about 70% or greater, about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.2% or greater, about 99.4% or greater, about 99.5% or greater, about 99.6% or greater, about 99.7% or greater, about 99.8% or greater, about 99.9% or greater, about 99.95% or greater, or 100% regioselectivity to 3-GOSA of the total geranyl-orsellinate (3-GOSA plus 5-GOSA) as determined in an in vitro enzymatic reaction using purified prenyltransferase variant.

Accordingly, of the geranyl-orsellinate reaction product, 5-GOSA is in an amount of less than about 10% (wt), less than about 9% (wt), less than about 8% (wt), less than about 7% (wt), less than about 6 (wt), less than about 5% (wt), less than about 4% (wt), less than about % (wt), less than about 2% (wt), less than about 1% (wt), less than about 0.8% (wt), less than about 0.6% (wt), less than about 0.5% (wt), less than about 0.4% (wt), less than about 0.3% (wt), less than about 0.2% (wt), less than about 0.1% (wt), less than about 0.05% (wt) or 0.0% (wt). In view of the improved regioselectivity of the prenyltransferase variants, the disclosure also provides compositions that are enriched for 3-GOSA, and derivatives thereof, such as pharmaceutical and non-pharmaceutical compositions having 90% or greater 3-GOSA as described herein, or other desired derivatives thereof.

In some embodiments, non-natural prenyltransferases with one or more variant amino acids as describe herein are enzymatically capable of providing regioselectivity to cannabigerol (CBG; 2-GOL) instead of the 4-GOL isomer, formed after reacting olivetol and GPP. In some embodiments the non-natural prenyltransferases with one or more variant amino acids providing an amount of regioselectivity to 2-GOL of about 60% or greater, about 70% or greater, about 80% or greater, about 85% or greater, about 90% or greater, about 91% or greater, about 92% or greater, about 93% or greater, about 94% or greater, about 95% or greater, about 96% or greater, about 97% or greater, about 98% or greater, about 99% or greater, about 99.2% or greater, about 99.4% or greater, about 99.5% or greater, about 99.6% or greater, about 99.7% or greater, about 99.8% or greater, about 99.9% or greater, about 99.95% or greater, or 100% regioselectivity to 2-GOL of the total cannabigerol isomers (2-GOL plus 4-GOL) as determined in an in vitro enzymatic reaction using purified prenyltransferase variant.

Accordingly, of the GPP—olivetol reaction product, 4-GOL is in an amount of less than about 10% (wt), less than about 9% (wt), less than about 8% (wt), less than about 7% (wt), less than about 6 (wt), less than about 5% (wt), less than about 4% (wt), less than about % (wt), less than about 2% (wt), less than about 1% (wt), less than about 0.8% (wt), less than about 0.6% (wt), less than about 0.5% (wt), less than about 0.4% (wt), less than about 0.3% (wt), less than about 0.2% (wt), less than about 0.1% (wt), less than about 0.05% (wt) or 0.0% (wt). In view of the improved regioselectivity of the prenyltransferase variants, the disclosure also provides compositions that are enriched for 2-GOL, and derivatives thereof, such as pharmaceutical and non-pharmaceutical compositions having about 90% or greater 2-GOL as described herein, or other desired derivatives thereof.

As described herein, the non-natural prenyltransferases of the disclosure can include one amino acid variation, two amino acid variations, three amino acid variations, four amino acid variations, five amino acid variations, or more than five amino acid variations, from a wild type prenyltransferase template sequence. The variation(s) can be any single or combinations as described herein. Optional variations, other than those described herein, can be used with any single or combinations as described herein, wherein the optional variations are not detrimental to the desired activity of the prenyltransferase variants. Exemplary optional variations include those such as conservative amino acid substitutions that do not considerably alter protein properties.

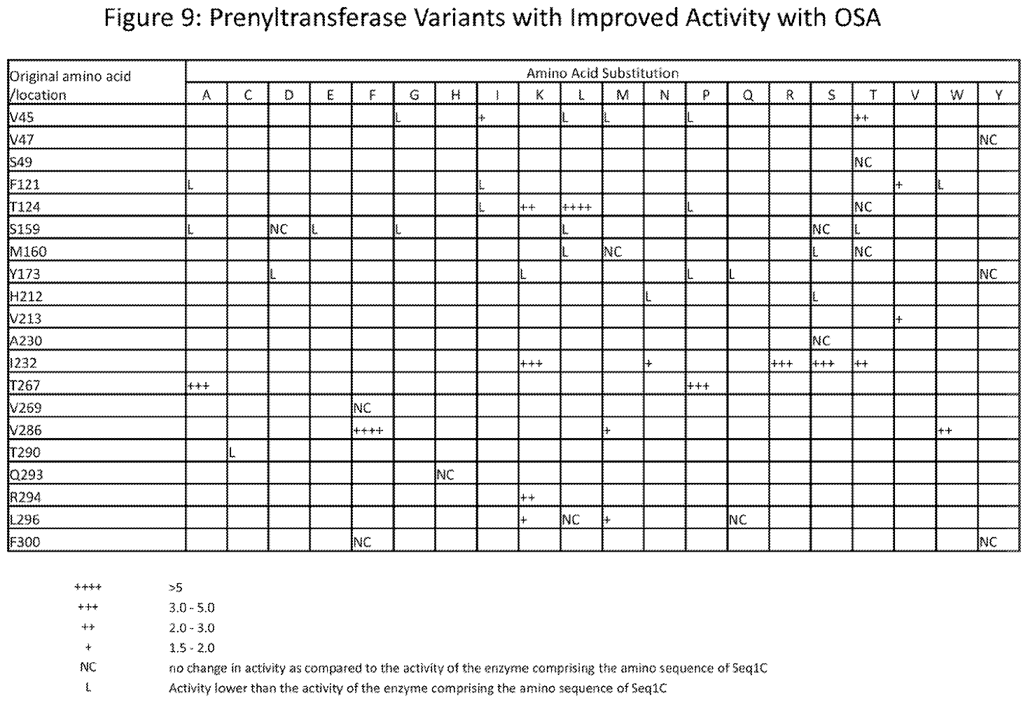

FIGS. 8 and 9 and the tables therein list positions of amino acids mutations providing increased enzymatic activity relative to the starting template. In particular, the triple variant Seq1C was used as a starting template for the introduction of further variation(s). The Seq1C variant is based on SEQ ID NO:1 having Q159S, S212H, and Y286V variant amino acids. The Seq1C template already provides a>300-fold enzymatic activity increase for conversion of OLA to CBGA over the wild type prenyltransferase enzyme (SEQ ID NO: 1). Results of the mutagenesis procedures revealed a number of further amino acid variations along the Seq1C prenyltransferase template showing improved formation of cannabigerolic acid (CBGA) from geranyl pyrophosphate and olivetolic acid, or improved formation of cannabigerorcinic acid (CBGOA) from orsellinic acid (OSA) and geranyl diphosphate (GPP). The mutations are described with reference to the numbering of amino acid positions in SEQ ID NO: 1 from which Seq1C was derived. The variant location and identities as set forth in this table, used in combination with the alignments shown in FIG. 5, can be used to introduce the variant amino acids into any other soluble prenyltransferase sequence that can be aligned with SEQ ID NO:1.

Site-directed mutagenesis or sequence alteration (e.g., site-specific mutagenesis or oligonucleotide-directed) can be used to make specific changes to a target prenyltransferase DNA sequence to provide a variant DNA sequence encoding prenyltransferase with the desired amino acid substitution. As a general matter, an oligonucleotide having a sequence that provides a codon encoding the variant amino acid is used. Alternatively, artificial gene sequence of the entire coding region of the variant prenyltransferase DNA sequence can be performed as preferred prenyltransferase targeted for substitution are generally less than 400 amino acids long.

Exemplary techniques using mutagenic oligonucleotides for generation of a variant prenyltransferase sequence include the Kunkel method which may utilize a prenyltransferase gene sequence placed into a phagemid. The phagemid in E. coli produces prenyltransferase ssDNA which is the template for mutagenesis using an oligonucleotide which is a primer extended on the template.

Depending on the restriction enzyme sites flanking a location of interest in the prenyltransferase DNA, cassette mutagenesis may be used to create a variant sequence of interest. For cassette mutagenesis, a DNA fragment is synthesized inserted into a plasmid, cleaved with a restriction enzyme, and then subsequently ligated to a pair of complementary oligonucleotides containing the prenyltransferase variant mutation. The restriction fragments of the plasmid and oligonucleotide can be ligated to one another.

Another technique that can be used to generate the variant prenyltransferase sequence is PCR site directed mutagenesis. Mutageneic oligonucleotide primers are used to introduce the desired mutation and to provide a PCR fragment carrying the mutated sequence. Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as DpnI which is specific for methylated DNA.

An expression vector or vectors (“an expression construct”) can be constructed to include one or more variant prenyltransferase encoding nucleic acids as exemplified herein operably linked to expression control sequences functional in the host organism. Expression vectors applicable for use in the microbial host organisms provided include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Exogenous variant prenyltransferase-encoding nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “isolated” when used in reference to a microbial organism is intended to mean an organism that is substantially free of at least one component that the referenced microbial organism is found with in nature. The term includes a microbial organism that is removed from some or all components as it is found in its natural environment. The term also includes a microbial organism that is removed from some or all components as the microbial organism is found in non-naturally occurring environments.

In some aspects the prenyltransferase variant gene is introduced into a cell with a gene disruption. The term “gene disruption,” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions. The phenotypic effect of a gene disruption can be a null mutation, which can arise from many types of mutations including inactivating point mutations, entire gene deletions, and deletions of chromosomal segments or entire chromosomes. Specific antisense nucleic acid compounds and enzyme inhibitors, such as antibiotics, can also produce null mutant phenotype, therefore being equivalent to gene disruption.

A metabolic modification refers to a biochemical reaction that is altered from its naturally occurring state. Therefore, microorganisms may have genetic modifications to nucleic acids encoding metabolic polypeptides, or functional fragments thereof. Exemplary metabolic modifications are disclosed herein.

The microorganisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

A variety of microorganism may be suitable for incorporating the variant prenyltransferase, optionally with one or more other transgenes. Such organisms include both prokaryotic and eukaryotic organisms including, but not limited to, bacteria, including archaea and eubacteria, and eukaryotes, including yeast, plant, insect, animal, and mammal, including human. Exemplary species are reported in U.S. application Ser. No. 13/975,678 (filed Aug. 26, 2013), which is incorporated herein by reference, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain embodiments, suitable organisms include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium Ijungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatas, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis stn. PCC 6803, Syntrophobacter fumaroxidans, Thauera aromatica, Thermoanaerobacter sp. X514, Thermococcus kodakanaensis, Thermococcus litonalis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

In exemplary embodiments, the engineered cell comprising the non-natural prenyltransferase further comprises an olivetolic acid pathway, such as an olivetolic acid pathway that includes polyketide synthase/olivetol synthase, olivetic acid cyclase, or both. In exemplary embodiments, the engineered cell further comprises a divarinolic acid (DVA) pathway or an orsellinic acid (OSA) OSA pathway. In exemplary embodiments, the engineered cell further comprises a geranyl pyrophosphate (GPP) pathway, such as one that includes geranyl pyrophosphate synthase. In exemplary embodiments, the GPP pathway comprises a mevalonate (MVA) pathway, a MEP pathway, a non-MEP, non-MVA pathway, or a combination of two or more pathways. In exemplary embodiments, the engineered cell further comprises a cannabinoid synthase.

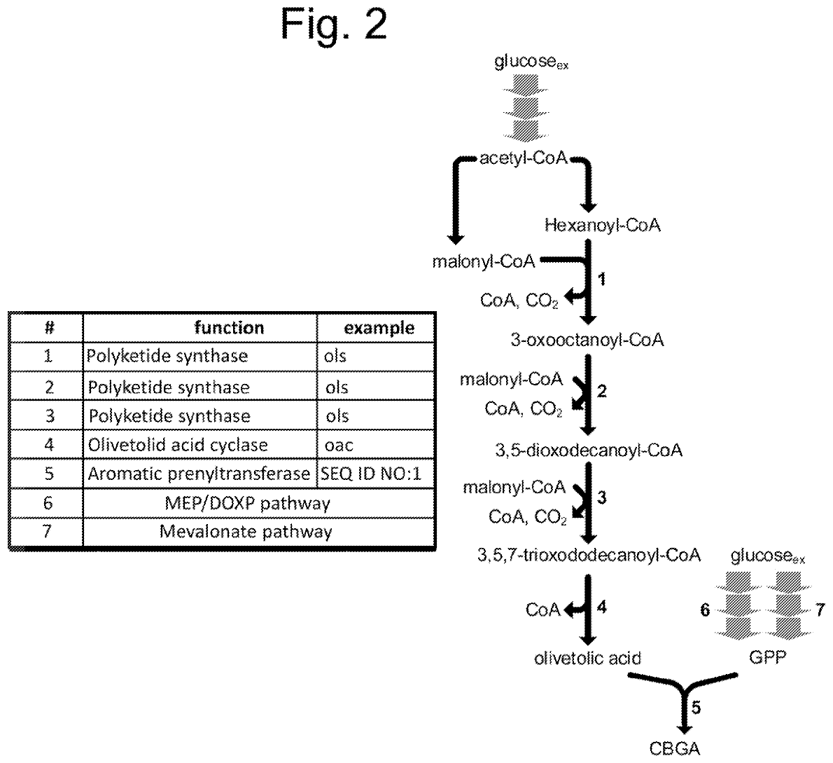

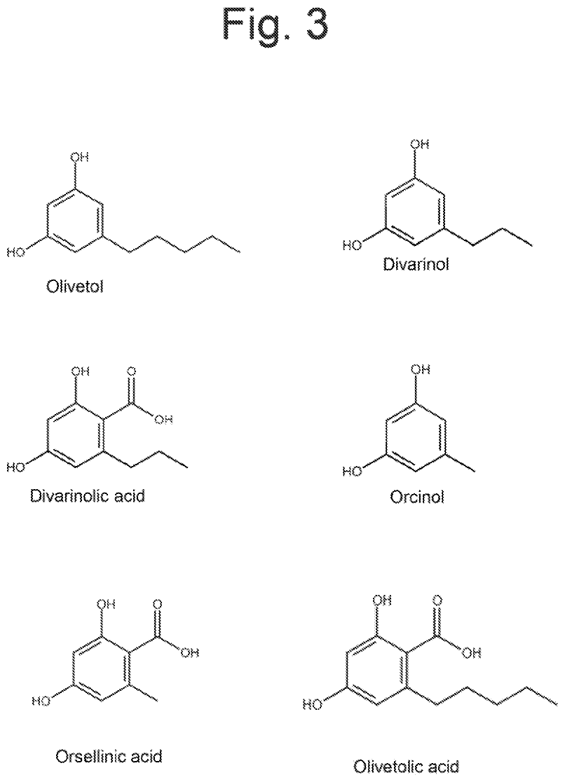

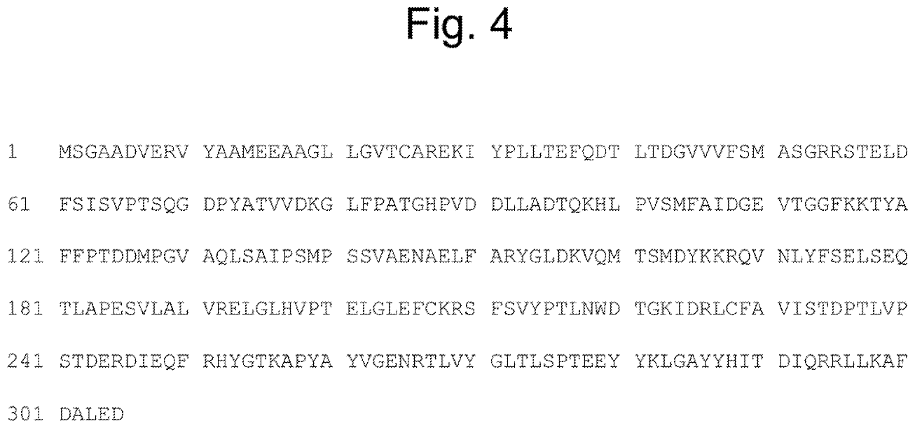

FIG. 2 shows exemplary pathways to CBGA formation from hexanoyl-CoA, and geranyl diphosphate. In some cases, the engineered cell of the disclosure can utilize hexanoyl-CoA that is produced from a cellular fatty acid biosynthesis pathway. For example, hexanoyl-CoA can be formed endogenously via reverse beta-oxidation of fatty acids.

In other embodiments, the engineered cell can further include hexanoyl-CoA synthetase, such as expressed on a transgene. Exemplary hexanoyl-CoA synthetase genes include enzymes endogenous to bacteria, including E. coli, as well as eukaryotes, including yeast and C. sativa (see for example Stout et al., Plant J., 2012; 71:353-365).

FIG. 2 also shows pathway formation of malonyl-CoA, which is used for the formation of olivetolic acid along with hexanoyl-CoA. Endogenous malonyl-CoA formation can be supplemented by formation from acetyl CoA using overexpression of acetyl-CoA carboxylase. Accordingly, the engineered cell can further include acetyl-CoA carboxylase, such as expressed on a transgene or integrated into the genome.

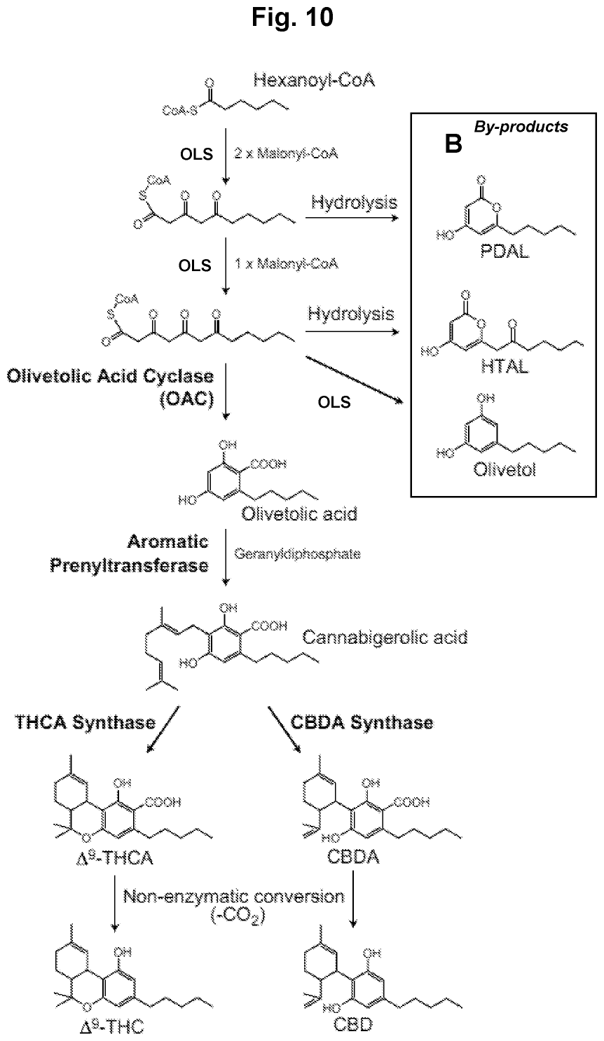

Similarly, FIG. 10 shows exemplary pathways to CBGA formation from malonyl-CoA, hexanoyl-CoA, and geranyl diphosphate. By-products of PDAL, HTAL, and olivetol in the OLS and OAC pathway and prior to formation of olivetolic acid, are shown. CBGA can then be modified to Δ⁹-THCA with THCA synthase, or to CBDA using CBDA synthase. Non enzymatic conversion (decarboxylation) can results in the products of Δ⁹-THC and CBD.

In other embodiments, the engineered cell that includes the variant prenyltransferase can further include hexanoyl-CoA synthetase, such as encoded by an exogenous nucleic acid. Exemplary hexanoyl-CoA synthetase genes include enzymes endogenous to bacteria, including E. coli, as well as eukaryotes, including yeast and C. sativa (see for example Stout et al., Plant J., 2012; 71:353-365, which is incorporated by reference in its entirety). Endogenous malonyl-CoA formation can be supplemented by formation from acetyl CoA using overexpression of acetyl-CoA carboxylase. Accordingly, the engineered cell can further include acetyl-CoA carboxylase, such as expressed on a transgene or integrated into the genome.

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms. Exemplary enzymes are encoded by accABCD of E. coli (Davis et al, J Biol Chem 275:28593-8 (2000)), ACC1 of Saccharomyces cerevisiae and homologs (Sumper et al, Methods Enzym 71:34-7 (1981)) as shown in Table 1.

TABLE 1

| Protein | GenBank ID | GI Number | Organism |
|---|---|---|---|
| ACC1 | CAA96294.1 | 1302498 | Saccharomyces cerevisiae |
| KLLA0F06072g | XP_455355.1 | 50310667 | Kluyveromyces lactis |
| ACC1 | XP_718624.1 | 68474502 | Candida albicans |
| YALI0C11407p | XP_501721.1 | 50548503 | Yarrowia lipolytica |
| ANI_1_1724104 | XP_001395476.1 | 145246454 | Aspergillus niger |
| accA | AAC73296.1 | 1786382 | Escherichia coli |
| accB | AAC76287.1 | 1789653 | Escherichia coli |
| accC | AAC76288.1 | 1789654 | Escherichia coli |
| accD | AAC75376.1 | 1788655 | Escherichia coli |

FIG. 2 also shows polyketide synthase converts hexanoyl-CoA to olivetolic acid through poly-β-keto intermediates. Accordingly, the engineered cell can further include polyketide synthase, such as expressed on a transgene or integrated into the genome. The engineered cell can further include olivetolic acid cyclase (oac), to convert 3,5,7-trioxododecanoyl-CoA to olivetolic acid.

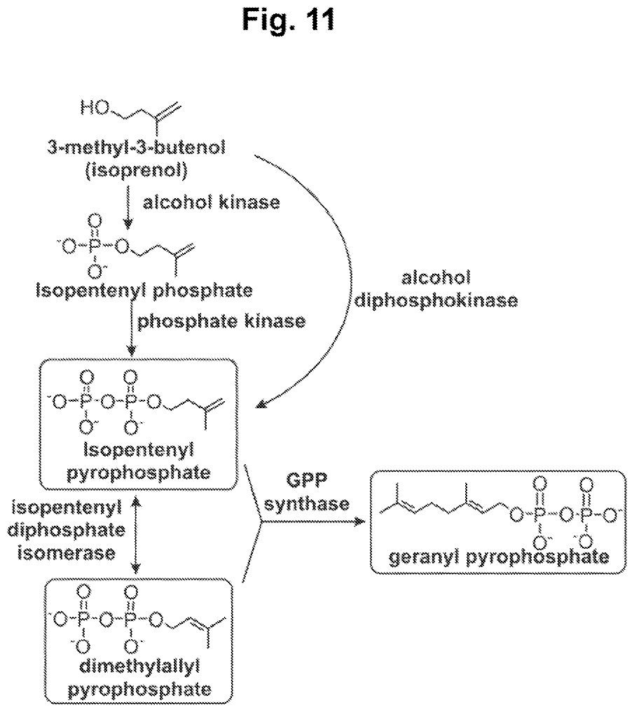

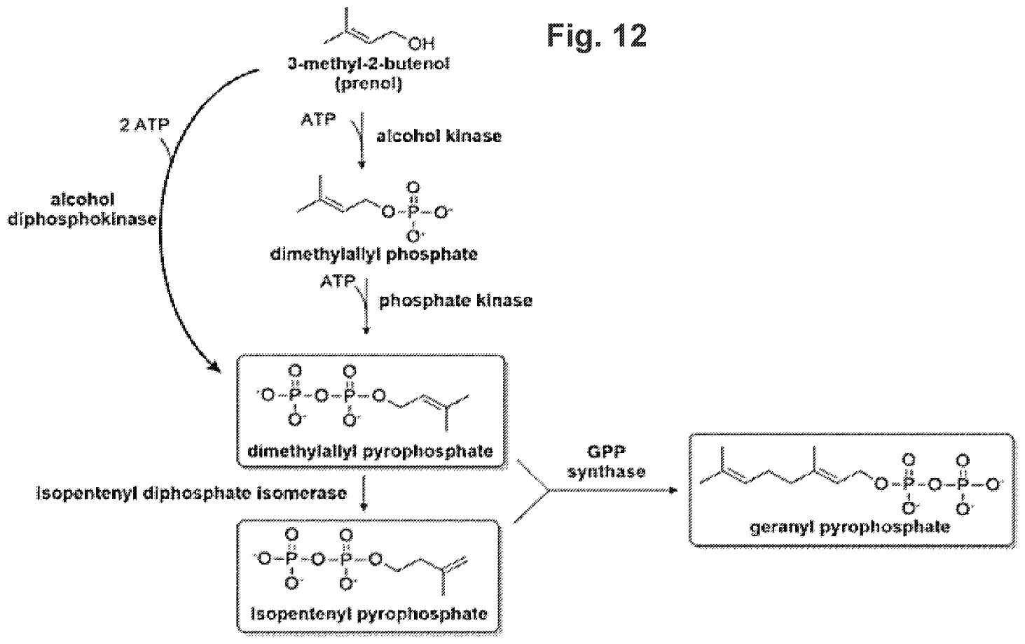

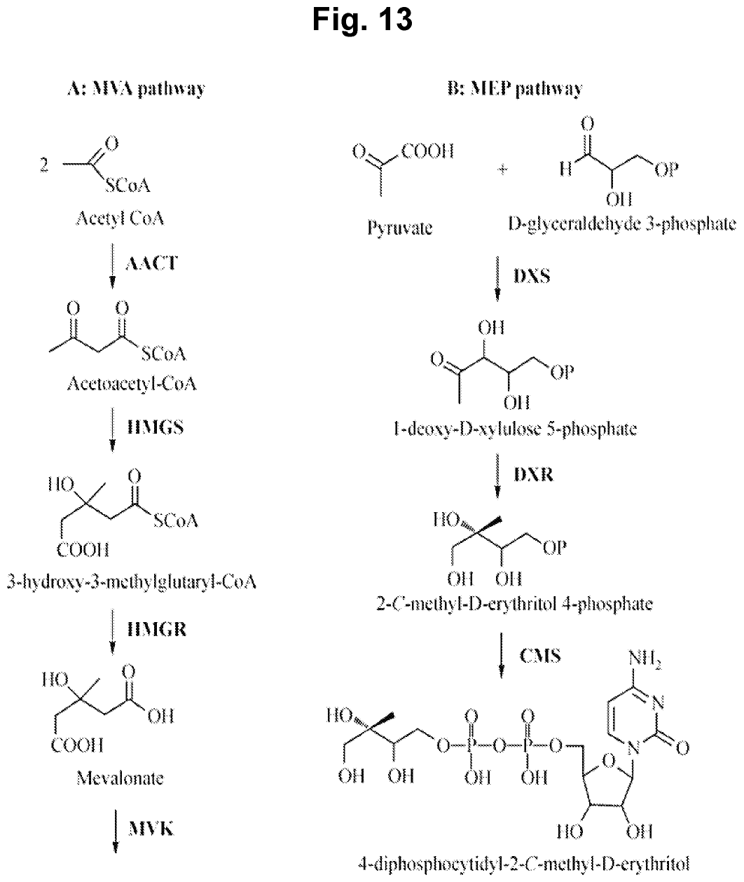

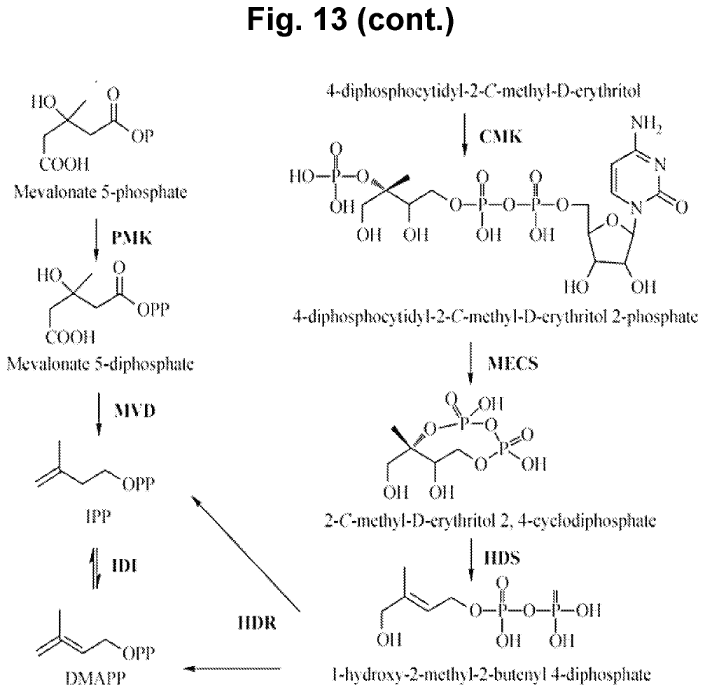

In some embodiments, the engineered cell comprises enzymes for the geranyl pyrophosphate pathway. In some embodiments, the geranyl pyrophosphate pathway comprises geranyl pyrophosphate synthase. In some embodiments, the geranyl pyrophosphate pathway comprises a mevalonate (MVA) pathway, a non-mevalonate (MEP) pathway, an alternative non-MEP, nor MVA geranyl pyrophosphate pathway using isoprenol or prenol as a precursor, or a combination thereof. Various pathways for generating geranyl pyrophosphate are disclosed in PCT publication WO2017161041, which is incorporated herein by reference in its entirety. Exemplary alternative non-MEP, nor MVA geranyl pyrophosphate pathways using isoprenol or prenol as a precursor are shown in FIGS. 11 and 12, respectively. Exemplary MVA and MEP pathways are shown in FIG. 13. In some embodiments, the engineered cell further comprises an exogenous nucleic acid encoding geranyl pyrophosphate synthase.

In some embodiments, the engineered cell preferentially uses a 5-alkylbenzene-1,3-diol as an (alcohol) substrate instead of an acid derivative of an alkylbenzene-1,3-diol. The 5-alkylbenzene-1,3-diol can be reacted with GPP to form a 2-prenylated 5-alkylbenzene-1,3-diol. For example, reaction of olivetol and GPP promoted with the non-natural prenyltransferase variants of the disclosure can form cannabigerol (CBG; 2-GOL). Accordingly, formation of the acid derivative of an alkylbenzene-1,3-diol can be avoided in cell. To avoid formation of the acid derivative, the olivetolic acid cyclase (oac) gene can be excluded from the pathway, or can be deleted from the cell. Gagne, S. J. et al (PNAS, 109:12811-12816, 2012) describes a pathways utilizing hexanoyl-CoA which can be converted to olivetol using tetraketide synthase (TKS), or further to olivetolic acid by action of olivetolic acid cyclase (oac).

Optionally, the engineered cell can include one or more exogenous genes which allow the cell to grow on carbon sources the cell would not normally metabolize, or one or more exogenous genes or modifications to endogenous genes that allow the cell to have improved growth on carbon sources the cell normally uses. For example, WO2015/051298 (MDH variants) and WO2017/075208 (MDH fusions) describe genetic modifications that provide pathways allowing to cell to grow on methanol; WO2009/094485 (syngas) describes genetic modifications that provide pathways allowing to cell to grow on synthesis gas.

As used herein, the term “bioderived” means derived from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the disclosure. A biobased or bioderived product is in contrast to a petroleum derived product, wherein such a product is derived from or synthesized from petroleum or a petrochemical feedstock.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, pannose, maltose, arabinose, cellobiose and 3-, 4-, or 5-oligomers thereof. Other carbon sources include alcohol carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, culture conditions include anaerobic or substantially anaerobic growth or maintenance conditions. Exemplary anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large scale culture procedures. Useful yields of the products can be obtained under anaerobic or substantially anaerobic culture conditions.

An exemplary growth condition for achieving, one or more cannabinoid product(s) includes anaerobic culture or fermentation conditions. In certain embodiments, the microbial organism can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6, or 7 days or more. Additionally, continuous culture can include 1 week, 2, 3, 4, or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

The culture medium at the start of fermentation may have a pH of about 5 to about 7. The pH may be less than 11, less than 10, less than 9, or less than 8. In other embodiments the pH may be at least 2, at least 3, at least 4, at least 5, at least 6, or at least 7. In other embodiments, the pH of the medium may be about 6 to about 9.5; 6 to about 9, about 6 to 8 or about 8 to 9.

Suitable purification and/or assays to test, e.g., for the production of 3-geranyl-olivetolate can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The 3-geranyl-olivetolate (CBGA) or other target molecules may be separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art. In one aspect disclosed herein are compositions comprising cannabinoids. Non-limiting examples of cannabinoids include tetrahydrocannabivarin (THCV), tetrahydrocannabivarinic acid (THCVA), cannabidivarin (CBDV), cannabidivarinic acid (CBDVA), cannabinol (CBN), cannabinolic acid (CBNA), cannabidiol (CBD), cannabidiolic acid (CBDA), cannabichromene (CBC), cannabichromenic acid (CBCA), cannabigerivarin (CBGV), cannabigerivarinic acid (CBGVA), cannabigerol (CBG), analogs, or derivatives thereof, or combinations thereof. In embodiments, in the composition the concentration of CBGA, CBG, CBGV, CBGVA; CBGOA, THCV, THCVA, CBD, CBDA, CBDV, CBDVA, CBN, CBNA, CBC, CBCA, analogs or derivatives thereof, or combinations thereof, is at least about is at least about: 50%, 55%, 60%, 65%, 70%, 75%, 80%, 85%, 90%, 95%, 97.5%, or 99% of the total weight of the composition. Accordingly, a by-product or combination of by-products (such as PDAL, HTAL, etc., as described herein), is present at a concentration of no more than about: 50%, 40%, 30%, 20%, 10%, 5%, 1%, 0.1%, 0.01%, 0.001%, or 0.0001%, of the total weight of the composition.

In some embodiments, cannabinoids, analogs, or derivatives thereof are produced by one or more enzymes or their variants, e.g., olivetol synthase, olivetolic acid cyclase, prenyltransferase, and cannabinoid synthases (e.g., CBDA synthase, THCA synthase). In some embodiments, analogs, or derivatives of CBGA are produced by prenyltransferase enzyme or variants of prenyltransferase enzyme disclosed in the present application.

In some embodiments, cannabinoids, analogs, or derivatives thereof are produced using malonyl-CoA and acyl-CoA by olivetol synthase or variants of olivetol synthase, olivetolic acid cyclase or its variants, prenyltransferase or its variants, and cannabinoid synthases.

In some embodiments, the acyl-CoA substrate has a following structure:

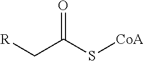

wherein R is a fatty acid side chain optionally comprising one or more functional and/or reactive groups as disclosed herein (i.e., an acyl-CoA compound derivative). In some embodiments, functional groups may include, but are not limited to, azido, halo (e.g., chloride, bromide, iodide, fluorine), methyl, alkyl (including branched and linear alkyl groups), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Functional and reactive groups may be optionally substituted with one or more additional functional or reactive groups.

In some embodiments, the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA.

In some embodiments, olivetol synthase catalyzes the formation of 3,5,7-trioxoacyl-CoA or a 3,5,7-trioxocarboxylate from malonyl-CoA and a acyl-CoA. In some embodiments, the acyl CoA molecules can be an acyl-CoA, aminoacyl-CoA (e.g., 2-aminoacetyl CoA, 3-aminopropionyl-CoA, 2-aminopropionyl-CoA, 4-aminobutyryl-CoA), hydroxyacyl-CoA (e.g., 2-hydroxypropionoyl-CoA, 3-hydroxybutyryl-CoA, hydroxyacetyl-CoA, hydroxypropionoyl-CoA, hydroxybutyryl-CoA), branched chain acyl-CoA (e.g., isobutyryl-CoA, 3-methylbutyryl-CoA), an aromatic acid CoA, for example, benzoic, chorismic, phenylacetic and phenoxyacetic acid CoA. Exemplary acyl-CoA include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA.

In some embodiments, CBGA, analogs, or derivatives thereof are essentially free of pesticides, heavy metals, other plant derived materials, or antibiotics. In some embodiments, the CBGA, a or derivatives thereof are at least about: 25%, 30%, 35%, 40%, 45%, 50%, 55%, 60%, 65%, 70%, 75%, 80%, 85%, 90%, 95%, 96%, 97%, 98%, 99%, 99.5%, 99.8% pure.

In some embodiments, the composition comprising cannabinoids, analogs, or derivatives thereof is a liquid, an engineered cell fermentation broth or cell culture medium, a cell free fermentation broth, or an engineered cell lysate. In some embodiments, the engineered cell, engineered cell extract, or engineered cell culture medium comprises olivetol or analogs and derivatives of olivetol, pentyl diacetic acid lactone (PDAL), hexanoyl triacetic acid lactone (HTAL), or lactone analog or derivatives thereof, or a combination thereof, at a concentration of no more than about 50% to about 0.0001%, no more than about 20% to about 0.001%, no more than about 10% to about 0.01% by weight of the engineered cell, engineered cell extract, or engineered cell culture medium.

In some embodiments, the molar ratio of CBGA to its analogs or derivatives is about 100:0, 99.9:0.1, 99.5:0.5, 99:1, 98:2, 97.5:2.5, 97:3, 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, 5:95, 2.5:97.5, 2:98, 1:99, 0.5:95, 0.1:99.9, 0.01:99.99.

In view of the regioselectivity of the prenyltransferase variants, the disclosure also provides compositions that are enriched for desired cannabinoids and derivatives thereof. In particular, the disclosure provides compositions enriched for CBGA (3-geranyl-olivetolate (3-GOLA)) and/or CBG compared to the undesired isomer, e.g. 5-GOLA or 4-GOL (decarboxylated 5-GOLA). Such enriched compositions include those that are pharmaceutical compositions as well as those that are used for non-pharmaceutical purposes, including medicinal purposes. Accordingly, in some embodiments, provided are compositions, such as pharmaceutical compositions or medicinal compositions, with CBGA and/or CBG that are about 90% or greater, about 91% or greater, about 92% or greater, about 93% or greater, about 94% or greater, about 95% or greater, about 96% or greater, about 97% or greater, about 98% or greater, about 99% or greater, about 99.2% or greater, about 99.4% or greater, about 99.5% or greater, about 99.6% or greater, about 99.7% or greater, about 99.8% or greater, about 99.9% or greater, about 99.95% or greater or even 100% 3-geranyl-olivetolate (3-GOLA) or its decarboxylated derivative CBG (2-GOL), of all geranyl-olivetolate compounds, including 5-GOLA and 4-GOL compounds, which can be less desirable when present in various compositions.

### Examples

### Seq1C Template for Second Generation Prenyltransferase Variants

Second generation prenyltransferase variants of the disclosure were generated and identified by introducing variant amino acids into template Seq1C (SEQ ID NO: 16, which is SEQ ID NO:1 having Q159S, S212H, and Y286V variant amino acids). The non-natural prenyltransferase triple variant (Seq1C) template is described in commonly assigned International Application No. PCT/US2019/021448 (filed Mar. 8, 2019; Noble, M., which is incorporated by reference in its entirety). Enzyme activity of the Seq1C triple variant (obtained from a previously frozen stock, see below) was shown to be 300-fold greater for conversion of OLA to CBGA, and greater 100-fold for conversion of OSA to CBGOA over the wild type prenyltransferase (PT) enzyme (SEQ ID NO: 1). The activities of Seq1C to various substrates are shown in Table 2.

For freshly purified enzymes, Seq ID 1C showed a >300-fold improvement over wild-type enzyme on OLA. However, in subsequent studies using prenyltransferase enzymes that were previously frozen (stored frozen >1 year) which were directed at testing for activity on a range of substrates (OL, OSA, DVA), the previously frozen and thawed enzymes showed a lower fold improvement (67 fold on OLA). Accordingly, the stability effects of prenyltransferase enzyme preparations and lower activities were believed to be caused by the freeze/thaw cycle.

TABLE 2

| | | Olivetolic | Divarinolic | Orsellinic | Orsellinic | |
|---|---|---|---|---|---|---|
| | Substrate | acid | acid | acid | acid | Olivetol |
| | Product | CBGA | CBGVA | CBGOA | CBGOA | Cannabigerol |
| | | 3-GOLA | 3-GDVA | 3-GOSA | 5-GOSA | (CBG, 2-GOL) |
| PT variant | Mutations | Relative fold | Relative fold | Relative fold | Relative fold | Relative fold |
| | | increase | increase | increase | increase | increase |
| Seq1C | Q159S | >300 | 330 | >100 | 0 | 60 |
| | S212H | | | | | |
| | Y286V | | | | | |

### Description of Library Constructs and Strains

Mutant variants of prenyltransferase based on the Seq1C template were constructed as libraries on plasmid by single-site mutagenesis methods, using specific primers at the positions undergoing mutagenesis, amplifying fragments via PCR, and circularizing plasmid via Gibson ligation. A compressed-codon approach was used to eliminate codon redundancy to lower library size. Plasmid used was the low copy pZS* plasmid (Expressys), with expression of the prenyltransferase gene under control of a T7 promoter and lac operator. Plasmids harboring the mutant libraries of prenyltransferase genes were transformed into E. coli strain BL21(DE3) and plated onto Agar plates with suitable antibiotic selection. The resulting prenyltransferase protein includes a fusion to a 6× Histidine tag at the N-terminus. Active variants were identified by activity assay described below and sequenced.

### Cell Culture for Screening Homologs and Mutant Libraries

From both mutant library transformants and control transformants, single colonies were picked for growth into 384-well plates using Luria Bertani (LB) growth medium with suitable antibiotic. Following overnight growth, cultures were sub-cultured into fresh medium of LB with antibiotic and IPTG. Cells were then cultured with rapid shaking at 22° C. for 20 hours, followed by centrifugation to pellet cells and then supernatant discarded. Cells pellets were stored at −20° C. until ready for assay. Number of samples screened was approximately three times oversample based on calculation of total possible variants.

### High-Throughput Activity Assay

Cell pellets were thawed, and subjected to chemical lysis by SoluLyse (Genlantis) in the presence of protease inhibitor cocktail, 5 mM DTT, and nuclease and lysozyme. Assays were performed in 384-well plates in a total volume of 50 μl in 50 mM Tris, pH 7.5 buffer containing 250 mM geranyl pyrophosphate (Sigma-Aldrich) and 75 μM olivetolic acid (OLA) (Santa Cruz Biotechnology) and 5 mM magnesium chloride, or containing 250 μM geranyl pyrophosphate and 250 μM orsellinic acid (OSA) and 5 mM magnesium chloride. Reactions were initiated by addition of cell lysate then incubated for a measured period of up to 1 hour, (such that template-catalysed conversion of substrate is approx. 1% of total), quenched with acetonitrile to a final concentration of 65%, then centrifuged to pellet denatured protein. Supernatants were transferred to new 384-well plates for LCMS analysis of CBGA.

### LCMSMS Analysis of Prenyltransferase Activity in Cell Lysates

CBGA, CBGOA, and their isomers 5-GOLA and 5-GOSA were analyzed by LCMSMS methods on Shimadzu UHPLC system coupled with AB Sciex Q1RAP4500 mass spectrometer. Agilent Eclipse XDB C18 column (4.6×3.0 mm, 1.8 um) was used with 1-2 min gradient elution at 1 mL/min using water containing 0.1% ammonia acetate as mobile phase A and 90% methanol containing 0.1% ammonia acetate as mobile phase B. The LC column temperature was maintained at 45° C. Negative ionization mode was used for all the analytes. The isomer pairs were resolved with appropriate gradient elution. Compounds were identified by their LC retention times and MRM transitions specific to the compounds.

Enzymatic reactions conducted in cell lysates were first treated with 9 volumes of organic solvent (acetonitrile containing internal standards) to precipitate proteins. The supernatant was recovered and further diluted (if necessary) for LCMSMS analysis.

Results of enzyme assays for prenyltransferase variants using the OLA substrate and the OSA substrate are shown in FIGS. 8 and 9, respectively.

1. A non-natural prenyltransferase having 50% or greater identity to any one of SEQ ID NOs:1-16, comprising at least one amino acid variation as compared to a wild type prenyltransferase, wherein the at least one amino acid variation is selected from a variation at position:
45; 121, wherein the amino acid variation is 121V; 124, wherein the amino acid variation is selected from 124K and 124L; 160; 173; 212, wherein the amino acid variation is 212N; 232, wherein the amino acid variation is selected from 232K, 232N, 232R, and 232S; 267, wherein the amino acid variation is selected from 267A and 267P; 269, wherein the amino acid variation is 269F; 286, wherein the amino acid variation is 286W; 290; 294; 296, wherein the amino acid variation is selected from 296K, 296M, and 296Q; and 300, wherein the amino acid variation is 300Y; wherein the amino acid positions are relative to SEQ ID NO:1 or a corresponding amino acid position in any of SEQ ID NOs:2-16.

2. A non-natural prenyltransferase having 50% or greater identity to any one of SEQ ID NOs:1-15, comprising at least three amino acid variations as compared to a wild type prenyltransferase, wherein at least two of the amino acid variations are selected from variations at positions 159, 212, and 286, and at least one other amino acid variation is selected from a variation at position 45, 47, 49, 121, 124, 160, 173, 213, 230, 232, 267, 269, 290, 294, 296, and 300, the amino acid positions relative to SEQ ID NO:1 or a corresponding amino acid position in any of SEQ ID NOs:2-15.

3. A non-natural prenyltransferase of claim 2, wherein the one or more amino acid variation(s) at a position selected from the group consisting of 45, 121, 124, 160, 173, 212, 232, 267, 269, 286, 290, 294, 296, and 300 is selected from the group consisting of:
45I; 45T; 45S,
121V;
124K; 124L;
160L; 160V; 160I;
173D; 173K; 173P; 173Q; 173E; 173F;
212N;
232K; 232N; 232R; 232S;
267A; 267P;
269F;
286W;
290A; 290I; 290M; 290S
294K; 294H;
296K; 296M; 296Q; and
300Y.

4. (canceled)

5. The non-natural prenyltransferase of claim 2 comprising at least two amino acid variations selected from 159S, 212H, and 286V.

6. (canceled)

7. (canceled)

8. The non-natural prenyltransferase of claim 2 comprising one or more amino acid variation(s) selected from the group consisting of:
124S;
232T;
296I; and
300P, and 300I

9. (canceled)

10. The non-natural prenyltransferase of claim 2 comprising one or more one amino acid variation selected from
47S, 47N, 47G;
121L;
161R, 161H, 161S;
175H, 175K, 175R;
211H; 211N;
214H;
230S;
268Y;
269N;
284S;
285Y;
286F, 286L, 286M, 286P, 286T, 286V, 286I, 286A;
288V, 288I;
296I; and
293H, 293M, 293F, 293W, 293C, 293C, 293A, 293S, 293V, 293D, 293Y, 293E, 293I, and 293T.

11. The non-natural prenyltransferase of claim 1 enzymatically capable of regioselectively forming a 2-prenylated 5-alkylbenzene-1,3-diol or a 3-prenylated 2,4-dihydroxy 6-alkylbenzenoic acid from geranyl pyrophosphate and a 5-alkylbenzene-1,3-diol, or a 2,4-dihydroxy 6-alkylbenzenoic acid, optionally enzymatically capable of 90% or greater regioselectivity to the 2-prenylated 5-alkylbenzene-1,3-diol or the 3-prenylated 2,4-dihydroxy 6-alkylbenzenoic acid from geranyl pyrophosphate and a 5-alkylbenzene-1,3-diol, or a 2,4-dihydroxy 6-alkylbenzenoic acid.

12. (canceled)

13. The non-natural prenyltransferase of claim 1 having 90% or greater identity, or 95% or greater identity to SEQ ID NO:1 or to any one of SEQ ID NO:2-15.

14. (canceled)

15. (canceled)

16. The non-natural prenyltransferase of claim 1, wherein the non-natural prenyltransferase is enzymatically capable of
(a) greater than about 1.5-fold rate of formation of 3-geranyl-olivetolate (3-GOLA), analogs, derivatives, or combinations thereof from geranyl pyrophosphate (GPP) and olivetolic acid (OLA), analogs, derivatives, or combinations thereof, as compared to the corresponding wild type prenyltransferase;
(b) greater than about 1.5-fold rate of formation of 3-geranyl-orsellinate (3-GOSA from geranyl pyrophosphate (GPP) and orsellinic acid (OSA), as compared to the corresponding wild type prenyltransferase;
(c) greater than about 1.5-fold rate of formation of 3-geranyl-divarinolinate from geranyl pyrophosphate (GPP) and divarinolic acid as compared to the corresponding wild type prenyltransferase;
(d) greater than about 1.5-fold rate of formation of 2-geranyl-olivetol from geranyl pyrophosphate (GPP) and olivetol as compared to the corresponding wild type prenyltransferase;
(e) greater than about 1.5-fold rate of formation of 2-geranyl-orcinol from geranyl pyrophosphate (GPP) and orcinol as compared to the corresponding wild type prenyltransferase;
(f) greater than about 1.5-fold rate of formation of 2-geranyl-divarinol from geranyl pyrophosphate (GPP) and divarinol as compared to the corresponding wild type prenyltransferase; or
any combination of (a)-(f).

17. (canceled)

18. (canceled)

19. The non-natural prenyltransferase of claim 2 comprising at least three amino acid variations at the following positions relative to SEQ ID NO:1 or a corresponding amino acid location in any of SEQ ID NOs:2-15, the variations selected from:
(i) 45I, (ii) 159S, and (iii) 286V;
(i) 45T, (ii) 159S, and (iii) 286V;
(i) 121V, (ii) 159S, and (iii) 286V;
(i) 124K, (ii) 159S, and (iii) 286V;
(i) 124L, (ii) 159S, and (iii) 286V;
(i) 159S, (ii) 160L, and (iii) 286V;
(i) 159S, (ii) 160L, and (iii) 286V;
(i) 159S, (ii) 160S, and (iii) 286V;
(i) 159S, (ii) 173D, and (iii) 286V;
(i) 159S, (ii) 173K, and (iii) 286V;
(i) 159S, (ii) 173P, and (iii) 286V;
(i) 159S, (ii) 173Q, and (iii) 286V;
(i) 159S, (ii) 173Y, and (iii) 286V;
(i) 159S, (ii) 212H, and (iii) 286V;
(i) 159S, (ii) 230S, and (iii) 286V;
(i) 159S, (ii) 267P, and (iii) 286V;
(i) 159S, (ii) 286V, and (iii) 293H;
(i) 159S, (ii) 286V, and (iii) 294K;
(i) 159S, (ii) 286V, and (iii) 296K;
(i) 159S, (ii) 286V, and (iii) 296L;
(i) 159S, (ii) 286V, and (iii) 296M;
(i) 159S, (ii) 286V, and (iii) 296Q;
(i) 159S, (ii) 286V, and (iii) 296M;
(i) 159S, (ii) 286V, and (iii) 300F; and
(i) 159S, (ii) 286V, and (iii) 300Y.

20. The non-natural prenyltransferase of claim 2 comprising at least four amino acid variations at positions relative to SEQ ID NO:1 or a corresponding amino acid location in any of SEQ ID NOs:2-15, the variations selected from:
(i) 45I, (ii) 159S, (iii) 212H, and (iv) 286V;
(i) V45T, (ii) 159S, (iii) 212H, and (iv) 286V;
(i) 121V, (ii) 159S, (iii) 212H, and (iv) 286V;
(i) 124K, (ii) 159S, (iii) 212H, and (iv) 286V;
(i) 124L, (ii) 159S, (iii) 212H, and (iv) 286V;
(i) 159S, (ii) 160L, (iii) 212H, and (iv) 286V;
(i) 159S, (ii) 160L, (iii) 212H, and (iv) 286V;
(i) 159S, (ii) 160S, (iii) 212H, and (iv) 286V;
(i) 159S, (ii) 173D, (iii) 212H, and (iv) 286V;
(i) 159S, (ii) 173K, (iii) 212H, and (iv) 286V;
(i) 159S, (ii) 173P, (iii) 212H, and (iv) 286V;
(i) 159S, (ii) 173Q, (iii) 212H, and (iv) 286V;
(i) 159S, (ii) 173Y, (iii) 212H, and (iv) 286V;
(i) 159S, (ii) 212H, (iii) 213V, and (iv) 286V;
(i) 159S, (ii) 212H, (iii) 230S, and (iv) 286V;
(i) 159S, (ii) 212H, (iii) 267P, and (iv) 286V;
(i) 159S, (ii) 212H, (iii) 286V, and (iv) 293H;
(i) 159S, (ii) 212H, (iii) 286V, and (iv) 294K;
(i) 159S, (ii) 212H, (iii) 286V, and (iv) 296K;
(i) 159S, (ii) 212H, (iii) 286V, and (iv) 296L;
(i) 159S, (ii) 212H, (iii) 286V, and (iv) 296M;
(i) 159S, (ii) 212H, (iii) 286V, and (iv) 296Q;
(i) 159S, (ii) 212H, (iii) 286V, and (iv) 296M;
(i) 159S, (ii) 212H, (iii) 286V, and (iv) 300F; and
(i) 159S, (ii) 212H, (iii) 286V, and (iv) 300Y.

21. A nucleic acid encoding the non-natural prenyltransferase of claim 1, or an expression construct comprising said nucleic acid.

22. (canceled)

23. An engineered cell comprising the non-natural prenyltransferase of claim 1 or a nucleic acid or expression construct encoding the non-natural prenyltransferase of claim 1.

24. The engineered cell of claim 23 comprising
(a) an olivetolic acid pathway, the olivetolic acid pathway optionally comprising a polyketide synthase/olivetol synthase, wherein the polyketide synthase/olivetol synthase catalyzes condensation of acyl coenzyme A (CoA) and malonyl CoA; olivetic acid cyclase, or both;
(b) a divarinolic acid (DVA) pathway or an orsellinic acid (OSA) pathway;
(c) a geranyl pyrophosphate (GPP) pathway, the GPP pathway optionally comprising geranyl pyrophosphate synthase or geranylgeranyl pyrophosphate synthase, or wherein the GPP pathway optionally comprises a mevalonate (MVA) pathway, a MEP pathway, a non-MEP, non-MVA pathway; or
two or more of (a)-(c)

25-37. (canceled)

38. The engineered cell of claim 23 selected from the group consisting of bacteria, fungi, yeast, algae, and cyanobacteria, optionally wherein the engineered cell is a bacteria selected from the group consisting of Escherichia, Corynebacterium, Bacillus, Ralstonia, Zymomonas, and Staphylococcus.

39. (canceled)

40. A cell extract or cell culture medium comprising cannabigerolic acid (CBGA), tetrahydrocannabivarin (THCV), tetrahydrocannabivarinic acid (THCVA), cannabidivarin (CBDV), cannabidivarinic acid (CBDVA), cannabinol (CBN), cannabinolic acid (CBNA), cannabidiol (CBD), cannabidiolic acid (CBDA), cannabichromene (CBC), cannabichromenic acid (CBCA), cannabigerivarin (CBGV), cannabigerivarinic acid (CBGVA), cannabigerol (CBG), analogs, or derivatives thereof, or combinations thereof containing or derived from the engineered cell of claim 23.

41-43. (canceled)

44. A method for forming a prenylated aromatic compound, comprising contacting a substrate comprising a hydrophobic portion and an aromatic substrate with a non-natural prenyltransferase of claim 2, wherein contacting forms a prenylated aromatic compound.

45. The method of claim 43, wherein aromatic substrate is selected from the group consisting of olivetol, olivetolic acid, divarinol, divarinolic acid, orcinol, orsellinic acid, and analogs or derivatives thereof, or combinations thereof.

46. (canceled)

47. The method of claim 44 wherein the step of contacting occurs in the engineered cell that expresses the non-natural prenyltransferase.

48. (canceled)

49. (canceled)

50. A method of making a therapeutic composition including prenylated aromatic compound or a derivative thereof, comprising a step of including the prenylated aromatic compound or a derivative thereof obtained from the engineered cell of claim 23, in a therapeutic composition.

51-53. (canceled)
